SEQUENCE LISTING (20 sequences)

SEQ ID NO: 1 - DNA, 333 bp, Homo Sapiens
atgaggttcatgactctcctcttcctgacagctctggcaggagccctggtctgtgcctatgatccagaggccgcctctgccccaggatcggggaacccttgccatgaagcatcagcagctcaaaaggaaaatgcaggtgaagacccagggttagccagacaggcaccaaagccaaggaagcagagatccagccttctggaaaaaggcctagacggagcaaaaaaagctgtggggggactcggaaaactaggaaaagatgcagtcgaagatctagaaagcgtgggtaaaggagccgtccatgacgttaaagacgtccttgactcagtactatag

SEQ ID NO: 2 - DNA, 234 bp, Homo Sapiens
atgaggttcatgactctcctcttcctgacagctctggcaggagccctggtctgtgcctatgatccagaggccgcctctgccccaggatcggggaaccataaacaaatggattgtttacagctacagaagcccccttcagagactgccaaatttctgtcctcatccaccaacctgcctagaagagagaagctagtgccctctgcaaaacctccccacactagggggctggtataa

SEQ ID NO: 3 - DNA, 366 bp, Homo Sapiens
atgaggttcatgactctcctcttcctgacagctctggcaggagccctggtctgtgcctatgatccagaggccgcctctgccccaggatcggggaacccttgccatgaagcatcagcagctcaaaaggaaaatgcaggtgaagacccagggttagccagacaggcaccaaagccaaggaagcagagatccagccttctggaaaaaggcctagacggagcaaaaaaagctgtggggggactcggaaaactaggaaaagatgcagtcgaagatctagaaagcgtgggtaaaggtggggaagagaggttggtctttggggctcctgtgaatctaacctccatccctctgacttctgtgagccgtccatga

SEQ ID NO: 4 - DNA, 57 bp, Homo Sapiens
atgaggttcatgactctcctcttcctgacagctctggcaggagccctggtctgtgcc

SEQ ID NO: 5 - DNA, 90 bp, Homo Sapiens
tatgatccagaggccgcctctgccccaggatcggggaacccttgccatgaagcatcagcagctcaaaaggaaaatgcaggtgaagaccca

SEQ ID NO: 6 - DNA, 276 bp, Homo Sapiens
tatgatccagaggccgcctctgccccaggatcggggaacccttgccatgaagcatcagcagctcaaaaggaaaatgcaggtgaagacccagggttagccagacaggcaccaaagccaaggaagcagagatccagccttctggaaaaaggcctagacggagcaaaaaaagctgtggggggactcggaaaactaggaaaagatgcagtcgaagatctagaaagcgtgggtaaaggagccgtccatgacgttaaagacgtccttgactcagtactatag

SEQ ID NO: 7 - DNA, 33 bp, Homo Sapiens
gaaaatgcaggtgaagacccagggttagccaga

SEQ ID NO: 8 - DNA, 87 bp, Homo Sapiens
aaagctgtggggggactcggaaaactaggaaaagatgcagtcgaagatctagaaagcgtgggtaaaggagccgtccatgacgttaaa

SEQ ID NO: 9 - DNA, 138 bp, Homo Sapiens
agccttctggaaaaaggcctagacggagcaaaaaaagctgtggggggactcggaaaactaggaaaagatgcagtcgaagatctagaaagcgtgggtaaaggagccgtccatgacgttaaagacgtccttgactcagta

SEQ ID NO: 10 - DNA, 141 bp, Homo Sapiens
agccttctggaaaaaggcctagacggagcaaaaaaagctgtggggggactcggaaaactaggaaaagatgcagtcgaagatctagaaagcgtgggtaaaggagccgtccatgacgttaaagacgtccttgactcagtacta

SEQ ID NO: 11 - protein, 110 aa, Homo Sapiens
MRFMTLLFLTALAGALVCAYDPEAASAPGSGNPCHEASAAQKENAGEDPGLARQAPKPRKQRSSLLEKGLDGAKKAVGGLGKLGKDAVEDLESVGKGAVHDVKDVLDSVL

SEQ ID NO: 12 - protein, 77 aa, Homo Sapiens
MRFMTLLFLTALAGALVCAYDPEAASAPGSGNHKQMDCLQLQKPPSETAKFLSSSTNLPRREKLVPSAKPPHTRGLV

SEQ ID NO: 13 - protein, 121 aa, Homo Sapiens
MRFMTLLFLTALAGALVCAYDPEAASAPGSGNPCHEASAAQKENAGEDPGLARQAPKPRKQRSSLLEKGLDGAKKAVGGLGKLGKDAVEDLESVGKGGEERLVFGAPVNLTSIPLTSVSRP

SEQ ID NO: 14 - protein, 19 aa, Homo Sapiens
MRFMTLLFLTALAGALVCA

SEQ ID NO: 15 - protein, 30 aa, Homo Sapiens
YDPEAASAPGSGNPCHEASAAQKENAGEDP

SEQ ID NO: 16 - protein, 91 aa, Homo Sapiens
YDPEAASAPGSGNPCHEASAAQKENAGEDPGLARQAPKPRKQRSSLLEKGLDGAKKAVGGLGKLGKDAVEDLESVGKGAVHDVKDVLDSVL

SEQ ID NO: 17 - protein, 11 aa, Homo Sapiens
ENAGEDPGLAR

SEQ ID NO: 18 - protein, 29 aa, Homo Sapiens
KAVGGLGKLGKDAVEDLESVGKGAVHDVK

SEQ ID NO: 19 - protein, 47 aa, Homo Sapiens
SSLLEKGLDGAKKAVGGLGKLGKDAVEDLESVGKGAVHDVKDVLDSV

SEQ ID NO: 20 - protein, 48 aa, Homo Sapiens
SSLLEKGLDGAKKAVGGLGKLGKDAVEDLESVGKGAVHDVKDVLDSVL

(19) United States
(12) Patent Application Publication
(10) Pub. No.: US 2013/0324476 A1
(43) Pub. Date: Dec. 5, 2013

(54) COSMETIC USE OF DERMICIDIN, AND ANALOGUES OR FRAGMENTS THEREOF

(71) Applicants: Caroline Delattre, La Brede (FR); Audrey Gueniche, Rueil-Malmaison (FR); Isabelle Castiel, Nice (FR); Dominique Bernard, Vanves (FR)

(72) Inventors: Caroline Delattre, La Brede (FR); Audrey Gueniche, Rueil-Malmaison (FR); Isabelle Castiel, Nice (FR); Dominique Bernard, Vanves (FR)

(73) Assignee: L'OREAL, Paris (FR)

(21) Appl. No.: 13/824,674

(22) PCT Filed: Sep. 23, 2011
(86) PCT No.: PCT/IB2011/054190

§ 371 Date: Aug. 21, 2013

Related U.S. Application Data
(60) Provisional application No. 61/344,827, filed on Oct. 19, 2010.
(60) Provisional application No. 61/344,826, filed on Oct. 19, 2010.

(30) Foreign Application Priority Data
Sep. 24, 2010 (FR) 1057690
Sep. 24, 2010 (FR) 1057699

(51) Int. Cl. G01N 33/68; A61Q 19/08; A61Q 19/00; A61K 8/64; C07K 14/47; C07K 7/08

(52) U.S. Cl. CPC G01N 33/6881; C07K 14/4723; C07K 7/08; A61Q 19/00; A61Q 19/007; A61K 8/64; A61Q 19/08
USPC 514/18.8; 530/350; 530/324; 530/327; 530/328; 514/44 R; 536/23.5; 435/7.1; 435/6.13

(57) ABSTRACT

The present invention relates, in particular, to the use of a sequence of amino acids of dermicidin or an analogue or fragment thereof, and at least one sequence of nucleic acids coding said sequence as a biomarker for ageing skin and/or the signs of skin ageing which are possibly associated with skin dryness.

The present invention relates to the field of biomarkers for the skin, and more particularly for aged skin, which may or may not be associated with dryness of the skin, and also to the use thereof as targets or cosmetic active agents.

The epidermis, the superficial part of the skin, is a tissue in which the cells are joined together and interlinked with one another and lie on a basal membrane. Said epidermis forms an outer coating comprising sebaceous or sweat glands, and hair follicles.

More specifically, the epidermis is a structure of which the homeostasis is the result of the processing of a finely regulated set of intracellular and extracellular signals acting at all steps of cell proliferation, migration and differentiation and of the synthesis of the various extracellular matrix components. These signals can, in particular, result from the action of factors produced by the keratinocytes.

The epidermis is conventionally divided into a basal layer of keratinocytes containing, in particular, skin stem cells and constituting the germinal layer of the epidermis, a spinous layer constituted of several layers of polyhedral cells positioned on the basal layer, a “granular” layer comprising one to three layers “of flattened cells” containing distinct cytoplasmic inclusions, keratohyalin granules, and finally an assembly of upper layers known as the horny layer (or stratum corneum), constituted of keratinocytes at the terminal stage of their differentiation, known as corneocytes.

The stratum corneum, the most outer part of the skin which provides the barrier function between the organism and the environment. and the hair shaft, which is the emergent part of the hair follicle that constitutes the head of hair, both represent the result of the keratinocyte differentiation process. Epidermal differentiation follows a process of maturation in which basal layer keratinocytes differentiate and migrate so as to result in the formation of corneocytes, dead cells that are completely keratinized. This differentiation is the result of perfectly coordinated phenomena which will result in the maintaining of epidermal homeostasis and give the skin a healthy, young, luminous and smooth appearance.

During aging, many physiological modifications of the skin, resulting from a dysfunction of epidermal homeostasis, and in particular from a dysfunction of epithelial differentiation of keratinocytes and/or of proteoglycan synthesis, occur.

The modifications of epidermal homeostasis which occur during aging result mainly in a decrease in keratinocyte differentiation causing a deficit in the protein matrix of horny cells, through an increase in metalloproteinases, and in their extracellular matrix-degrading activity, and also in a decrease in the synthesis of the various glucosaminoglycans.

During skin aging, these modifications generally result in the appearance of a more marked microrelief of the skin, or even of fine lines, and finally in the occurrence of deep wrinkles, a loss of elasticity, a coarse feel, and dryness of the skin. Histologically, a flattening of the dermal-epidermal junction and a decrease in the thickness of the dermis and of the epidermis are observed.

Skin moisturization problems, and in particular dryness of the skin, can also often be observed with age.

What is more, many external factors can also reinforce the drying of the skin or worsen this state. Among these factors, mention may be made of climatic conditions such as cold or wind, sunlight, or exposure to certain chemical or therapeutic agents.

In physiological terms, dry skin is often associated with a decrease in the degree of skin moisturization and also a modification of the process of maturation of the stratum corneum, the most visible sign of which is the appearance of squamae at the surface of the skin. In sensory terms, dry skin may be characterized by a sensation of tautness and/or skin tension.

The collagen and glycosaminoglycan content of the skin is also decreased and the barrier function of aged skin may be modified.

Many epidermal factors, the expression, biological activity or maturation of which are modified, decreased or increased, are known to be involved, directly or indirectly, in the occurrence and the manifestation of aged skin or of the various associated signs of skin aging, such as dryness of the skin.

These factors can be used as biomarkers for the skin, as screening targets, or even as cosmetic active agents.

However, there still remain many unknowns regarding the intimate mechanism and regarding all the factors involved in skin aging, which may or may not be associated with dehydration.

Thus, there remains a need to identify novel biomarkers for the skin which are in particular capable of characterizing aged skin or signs of skin aging, in particular aged and dry skin or signs of dryness of the skin associated with aged skin.

There also remains a need to have novel biomarkers which make it possible to characterize a state of the skin, and in particular aged skin or signs of skin aging, in particular aged and dry skin or signs of dryness of the skin associated with aged skin.

There still remains a need to have novel tools for screening for active agents or physical treatments which are suitable for skin care, and which are more particularly of use for preventing and/or treating aged skin or signs of skin aging, in particular aged and dry skin or signs of dryness of the skin associated with aged skin.

There still remains a need to have novel active agents or novel treatments for preventing and/or treating aged skin or the signs of skin aging, in particular aged and dry skin or signs of dryness of the skin associated with aged skin.

There still remains a need to have novel cosmetic targets for skincare, and in particular for preventing and/or treating aged skin or the signs of skin aging, in particular aged and dry skin or signs of dryness of the skin associated with aged skin.

There also remains a need to identify novel biomarkers for dry skin or for the signs of dryness of the skin.

There also remains a need to have novel biomarkers which make it possible to characterize dry skin or signs of dryness of the skin.

There is still a need to have novel tools or targets for screening for active agents or physical treatments which are suitable for the care of dry skin, and more particularly which are of use for preventing and/or treating dry skin or the signs of dryness of the skin.

There is still a need to have novel tools or targets for screening for active agents or physical treatments for promoting or reinforcing skin moisturization.

There is still a need to have novel active agents or novel treatments for promoting and/or reinforcing skin moisturization.

There is still a need to have novel active agents or novel treatments for preventing and/or treating dry skin or the signs of dryness of the skin.

There is still a need to have novel cosmetic targets for skincare, especially for promoting and/or reinforcing skin moisturization and in particular for preventing and/or treating dry skin or the signs of dryness of the skin.

It is an object of the present invention to satisfy these needs.

Thus, according to one of its first subjects, the present invention relates to the use (i) of at least one amino acid sequence encoded by a nucleic acid sequence represented by SEQ ID No.: 1, or of an analog or fragment of said amino acid sequence, or (ii) of said nucleic acid sequence, as a biomarker for a state of the skin.

Preferably, a biomarker of the invention is a biomarker for aged skin and/or for the signs of skin aging, which may or may not be associated with dryness of the skin.

A biomarker of the invention can also be a biomarker for dry skin or for signs of dryness of the skin.

Thus, the use (i) of at least one amino acid sequence encoded by a nucleic acid sequence represented by SEQ ID No.: 1, or of an analog or fragment of said amino acid sequence, or (ii) of said nucleic acid sequence, as a biomarker for a moisturization state of the skin, and in particular dry skin and/or the signs of dryness of the skin, is also described.

For the purposes of the invention, the term “biomarker” means a molecule or the activity of a molecule, the presence, the content or the degree of activity of which is characteristic of a biological, physiological or pathological process, or of the impact or the effect induced by the administration of an active agent or of a physical treatment on such a process.

For the purposes of the invention, the term “skin” is intended to denote the whole of the epidermis of the human body, including the scalp and the lips. More particularly, the skin considered in the present invention is preferably the lips and the skin of the face, the neckline, the arms or the legs, and preferably the skin of the face and/or of the neckline.

Unexpectedly, during a differential proteomic study using the “iTRAQ” technology, the inventors have observed, from proteins extracted from varnish strippings of populations of different age classes that dermicidin (or DCD) proves to be a sensitive and specific biomarker for aged skin.

More specifically, the inventors have observed that the level of expression of dermicidin, and more particularly of peptides derived from dermicidin and identified by the sequences SEQ ID No.: 17 and SEQ ID No.: 18 are systematically decreased in aged skin compared with young skin.

In addition, unexpectedly, during a comparative proteomic study by Western blotting, the inventors have observed, from proteins extracted from varnish strippings of populations of different age classes exhibiting various degrees of skin moisturization, that dermicidin (or DCD) proves to be a sensitive and specific biomarker for dry skin.

Thus, the inventors have observed that the level of expression of dermicidin, and more particularly of a peptide identified by the sequence SEQ ID No.: 16, is systematically decreased in dry skin compared with normally moisturized skin. To the inventors' knowledge, this antimicrobial protein of the skin surface has never been classified as a variant during aging or during dehydration of the skin, and even less as one of the most relevant biomarkers emerging from studies of this type.

It is known that sweating decreases with age (Anderson and Kenney 1987; Kenney and Fowler 1988) and that dermicidin is a sudoriferous protein (Schittek, Hipfel et al., 2001). On the other hand, the experimental data obtained by the inventors show, unexpectedly, that there is a specific deficiency in expression and/or in maturation of DCD during skin aging, since said DCD is under-represented in the skin samples, even compared with other proteins of sudoriferous origin. Moreover, it may be emphasized that dryness of the skin is a complex phenomenon which is not systematically associated with a sudoriferous deficiency.

According to yet another of its subjects, the present invention relates to the use (i) of at least one amino acid sequence of the invention, or (ii) of a nucleic acid sequence of the invention, for screening for active agents or physical treatments capable of modulating the activity, the expression or the maturation of said amino acid sequence or of said nucleic acid sequence.

The active agents or the physical treatments screened may be more particularly intended for preventing and/or treating aged skin and/or the signs of skin aging, which may or may not be associated with dryness of the skin.

Also described is the use (i) of at least one amino acid sequence of the invention, or (ii) of a nucleic acid sequence of the invention, for screening for active agents or physical treatments capable of modulating the activity, the expression or the maturation of said amino acid sequence or of said nucleic acid sequence and intended for promoting and/or reinforcing skin moisturization.

Preferably, such active agents or physical treatments can be intended for preventing and/or treating dry skin and/or the signs of dryness of the skin.

For the purposes of the invention, the term “expression” means, with regard to an amino acid sequence, for example a protein or peptide, or to a nucleic acid sequence, for example an mRNA, its content or the variation of its content relative to a reference.

For the purposes of the invention, the term “maturation” means, with regard to an amino acid sequence, for example a protein or peptide, or to a nucleic acid sequence, for example an mRNA, the modifications which follow their synthesis in a cell environment. For example, in the case of an amino acid sequence, the term “maturation” means the post-translational modifications, such as glycosylation or farnesylation of certain amino acids, or the proteolytic steps resulting in the elimination of “signal” or “secretory” sequences or the release of sequences having particular biological properties. In the case of a nucleic acid sequence, the term “maturation” means, for example, the alternative splicing of an mRNA.

For the purposes of the invention, the term “activity” means, with regard to an amino acid sequence, for example a protein or peptide, the biological activity of the amino acid sequence, where appropriate after maturation, such as an enzymatic activity, an agonist or antagonist activity with respect to a receptor, an enzyme activating or inhibiting activity, a “structural” activity, or an antimicrobial activity.

For the purposes of the invention, the term “activity” means, with regard to a nucleic acid sequence, for example an mRNA, its translation.

According to another of its subjects, the present invention relates to the use (i) of at least one amino acid sequence of the invention, or (ii) of at least one nucleic acid sequence of the invention, for characterizing the efficacy of a cosmetic treatment for the skin.

Also described is the use (i) of at least one amino acid sequence of the invention, or (ii) of at least one nucleic acid sequence of the invention, for characterizing the efficacy of a cosmetic treatment for dry skin and/or for the signs of dryness of the skin.

According to one preferred embodiment, a cosmetic treatment of which the efficacy is characterized can be a treatment for aged skin and/or for the signs of skin aging, which may or may not be associated with dryness of the skin.

According to yet another of its subjects, the present invention relates to the cosmetic use of an effective amount (i) of at least one amino acid sequence of the invention, or (ii) of at least one nucleic acid sequence of the invention, or (iii) of at least one agent for modulating the activity, the expression or the maturation of said amino acid sequence or of said nucleic acid sequence, as an active agent for preventing and/or treating aged skin and/or the signs of skin aging, which may or may not be associated with dryness of the skin.

Also described is the cosmetic use of an effective amount (i) of at least one amino acid sequence of the invention, or (ii) of at least one nucleic acid sequence of the invention, or (iii) of at least one agent for modulating the activity, the expression or the maturation of said amino acid sequence or of said nucleic acid sequence, as an active agent for promoting and/or reinforcing skin moisturization.

Preferably, such a use can be devoted to preventing and/or treating dry skin and/or the signs of dryness of the skin.

For the purposes of the present invention, the term “effective amount” of a compound of the invention means an amount of this compound which is sufficient and necessary for obtaining a desired effect, and more particularly a cosmetic or care effect with regard to aged skin and/or to the signs of skin aging, which may or may not be associated with dryness of the skin. Likewise, an “effective amount” of a compound of the invention may be an amount of this compound which is sufficient and necessary to obtain a cosmetic or care effect with regard to moisturization of the skin, and in particular dry skin and/or the signs of dryness of the skin.

For the purposes of the invention, the term “preventing” means reducing the risk of occurrence or slowing down the occurrence of a given phenomenon, namely, in the present invention, aged skin and/or the signs of skin aging, which may or may not be associated with dryness of the skin. Likewise, for the purposes of the invention, the term “preventing” means reducing the risk of occurrence or slowing down the occurrence of dry skin and/or the signs of dryness of the skin.

According to yet another of its subjects, the present invention relates to the use of an effective amount (i) of at least one amino acid sequence of the invention, or (ii) of at least one nucleic acid sequence of the invention, or (iii) of at least one modulating agent of the invention, for preparing a multistratified epithelial cell model.

Preferably, a multistratified epithelial cell model, prepared according to the invention, can be a reconstructed skin model.

According to yet another of its subjects, the present invention relates to a method for characterizing a state of aged skin and/or signs of skin aging, which may or may not be associated with dryness of the skin, comprising at least the steps consisting in:

a) carrying out, in an isolated sample of skin, a qualitative or quantitative measurement of the expression, the maturation or the activity of said amino acid sequence or of said nucleic acid sequence, and

b) comparing said measurement carried out in step a) to a reference measurement.

Depending on the difference observed between the measurement obtained and the reference measurement, the skin will be described as normally young skin or as aged skin, or even aged and dry skin.

According to one preferred embodiment, a state of the skin under consideration in the invention is aged skin chosen from skin having undergone chronological aging and/or photoinduced aging.

Also described is a method for characterizing a moisturization state of the skin, and in particular dry skin and/or signs of dryness of the skin, comprising at least the steps consisting in:

a) carrying out, in an isolated sample of skin, a qualitative or quantitative measurement of the expression, the maturation or the activity of said amino acid sequence or of said nucleic acid sequence, and

b) comparing said measurement carried out in step a) to a reference measurement.

Depending on the difference observed between the measurement obtained and the reference measurement, the skin will be described as normally moisturized skin or as dry skin.

According to yet another of its subjects, the present invention relates to a method for screening for active agents or physical treatments capable of modulating the activity, the expression or the maturation of an amino acid sequence of the invention or of a nucleic acid sequence of the invention, comprising at least the steps consisting in:

a) placing said amino acid sequence or said nucleic acid sequence under conditions favorable to the activity, the expression or the maturation of said sequences,

b) bringing said amino acid sequence or said nucleic acid sequence into contact with at least one active agent to be tested, or exposing said amino acid sequence or said nucleic acid sequence to a physical treatment to be tested,

c) carrying out a qualitative or quantitative measurement of the expression, the maturation or the activity of said amino acid sequence or of said nucleic acid sequence, and

d) comparing said measurement to a reference measurement.

Depending on the difference observed between the measurement obtained and the reference measurement, the compound or the physical treatment screened will possibly be characterized as being of use for preventing and/or treating aged skin and/or the signs of skin aging, which may or may not be associated with dryness of the skin.

According to one preferred embodiment of the invention, the active agent(s) or the physical treatment(s) screened may be more particularly devoted to preventing and/or treating aged skin and/or the signs of skin aging, which may or may not be associated with dryness of the skin.

Also described is a method for screening for active agents or physical treatments capable of modulating the activity, the expression or the maturation of an amino acid sequence of the invention or of a nucleic acid sequence of the invention and intended for promoting and/or reinforcing the moisturization of dry skin, comprising at least the steps consisting in:

a) placing said amino acid sequence or said nucleic acid sequence under conditions favorable to the activity, the expression or the maturation of said sequences,

b) bringing said amino acid sequence or said nucleic acid sequence into contact with at least one active agent to be tested, or exposing said amino acid sequence or said nucleic acid sequence to a physical treatment to be tested,

c) carrying out a qualitative or quantitative measurement of the expression, the maturation or the activity of said amino acid sequence or of said nucleic acid sequence, and

d) comparing said measurement to a reference measurement.

According to one preferred embodiment, a method or use in accordance with the invention can be carried out in vivo, in vitro, or ex vivo, and even more preferably in vitro or ex vivo.

According to yet another embodiment, the present invention relates to an isolated peptide represented by an amino acid sequence chosen from SEQ ID No.: 17 or SEQ ID No.: 18, or an analog or fragment thereof.

The present invention has the advantage of providing a novel sensitive and specific biomarker for the skin, and in particular for aged skin or for the signs of skin aging, which may or may not be associated with dryness of the skin.

Likewise, the present invention has the advantage of providing a novel sensitive and specific biomarker for moisturization of the skin, and in particular dry skin or the signs of dryness of the skin.

The observation of the presence of dermicidin in the stratum corneum, and more particularly of peptides derived specifically from this protein, makes advantageously possible a quantitative or qualitative determination of the expression or of the activity of this protein, or the corresponding peptides, by simply taking a topical sample.

The sampling method may, for example, be a technique of stripping type, consisting in applying a portion of adhesive tape to the epidermis under consideration. When this adhesive tape is detached, a fraction of the skin surface is removed. After protein extraction, the latter can then be analyzed by conventional methods, such as ELISA immunoenzymatic assay or Western blot analysis, or more particularly by the iTRAQ differential proteomic method as defined hereinafter.

Likewise, the present invention has the advantage of being able to make available a novel biomarker suitable for screening for novel active agents or for novel physical treatments suitable for preventing and/or treating aged skin and/or the signs of skin aging, which may or may not be associated with dryness of the skin. Likewise, the present invention has the advantage of being able to make available a novel biomarker suitable for screening for novel active agents or for novel physical treatments that are of use for promoting and/or reinforcing skin moisturization. Preferably, such agents or treatment can be suitable for preventing and/or treating dry skin and/or the signs of dryness of the skin.

Likewise, according to another advantage, the present invention makes it possible to provide novel active agents suitable for preventing and/or treating aged skin and/or the signs of skin aging, which may or may not be associated with dryness of the skin.

Amino Acid and Nucleic Acid Sequences

Dermicidin or preproteolysin (DCD) is a protein of 110 amino acids (SEQ ID No.: 11) comprising a signal peptide of 19 amino acids (SEQ ID No.: 14), the gene of which is located on chromosome 12, locus 12q13.1. Two isoforms of this protein are registered, one shorter, isoform 1 (SEQ ID No.: 12), the other longer, isoform 2 (SEQ ID No.: 13). It is secreted in sweat (Schittek, Hipfel et al. 2001).

After cleavage of the signal peptide, dermicidin (DCD) is a protein of 90 amino acids according to the sequence SEQ ID No.: 16.

After proteolytic post-translational maturation, essentially by cathepsin D, which takes place in the sweat (Baechle, Flad et al., 2006), this protein precursor gives rise to DCD-1 (SEQ ID No.: 19) and to DCD-1L (SEQ ID No.: 20) and also to numerous peptides, the length of which ranges from 25 to 48 aa (Flad, Bogumil et al., 2002).

Unless otherwise indicated, the term “dermicidin” aims to denote in the present application the amino acid sequences represented by SEQ ID No.: 11, SEQ ID No.: 12, SEQ ID No.: 13, SEQ ID No.: 16, SEQ ID No.: 19 and SEQ ID No.: 20, possibly having undergone post-translational maturation.

According to one preferred embodiment, the term “dermicidin” is intended to denote more particularly the amino acid sequence represented by SEQ ID No.: 16.

Among the peptides derived from the maturation of dermicidin as described in US 2008/020976, some show antimicrobial activities against various microorganisms, such as Staphylococcus aureus, Escherichia coli, Enterococcus faecalis, or Candida albicans. The antimicrobial peptides are derived from the C-terminal part of the protein.

The N-terminal part of the protein (SEQ ID No.: 15) is constituted of a biologically active factor which promotes cell survival (Cunningham, Hodge et al. 1998).

This protein is expressed constitutively in the sweat gland (Rieg, Garb et al. 2004), and is involved in signaling pathways of PI3K/AKT/mTOR type particularly important in cell proliferation and survival (Moreira, Strauss et al. 2008). The decrease in DCD observed in the sweat of individuals suffering from atopy could partly explain their microbial susceptibility (Rieg, Steffen et al. 2005).

Some of the antimicrobial fragments and also fragments of the N-terminal part are capable of stimulating cytokine/chemokine production by keratinocytes or other cell types, implicating it in the regulation of skin immunity (Watchorn, Dowidar et al. 2005; Niyonsaba, Suzuki et al. 2009). It has recently been proposed to use DCD as a particularly sensitive, specific marker for the presence of traces of sweat by RT-PCR or by ELISA which can be used in the medicolegal field (Sakurada, Akutsu et al. 2009).

According to one embodiment, an amino acid sequence suitable for the invention can be encoded by a nucleic acid sequence represented by SEQ ID No.: 1, or be an analog or a fragment of this amino acid sequence.

The expression “analog of an amino acid sequence” in accordance with the invention is intended to denote any amino acid sequence having a sequence identity of at least 85%, preferably of at least 90%, and more preferentially of at least 95%, with said sequence, and a biological activity of the same nature.

The expression “biological activity of the same nature” with regard to an amino acid sequence according to the invention can mean the antimicrobial or cell survival and/or proliferation stimulation properties usually attributed to dermicidin. Preferably, the expression “biological activity of the same nature” with regard to an amino acid sequence according to the invention means the preventing and/or treating properties with regard to aged skin or the signs of skin aging, which may or may not be associated with dryness of the skin, or even with regard to dry skin and/or the signs of dryness of the skin.

The sequence identity can be determined by visual comparison or by means of any computer tool generally used in the field, such as the BLAST programs available on www.ncbi.nlm.nih.gov and used with the default parameters.

An analog in accordance with the invention may be a peptidomimetic agent.

An analog of an amino acid sequence of the invention can result from modifications resulting from mutation or variation in the sequences of the peptides according to the invention originating either from the deletion or the insertion of one or more amino acids, or from the substitution of one or more amino acids, or else from alternative splicing. Several of these modifications can be combined.

Advantageously, an analog of an amino acid sequence of the invention can comprise conservative substitutions compared with this amino acid sequence.

By way of example of mutations that can be considered in the present invention, mention may be made, nonexhaustively, of the replacement of one or more amino acid residues with amino acid residues having a similar hydropathic index without, however, substantially affecting the biological properties of the polypeptide. The hydropathic index is an index assigned to amino acids according to their hydrophobicity and their charge (Kyte et al. (1982), J. Mol. Biol., 157: 105).

An amino acid sequence or an analog thereof targeted by the present invention can be an amino acid sequence having undergone one or more post-translational maturation(s).

The term “post-translational maturation(s)” is intended to encompass all the modifications that an amino acid sequence is liable to undergo at the end of its synthesis in a cell, such as, for example, one or more phosphorylation(s), one or more thiolation(s), one or more acetylation(s), one or more glycosylation(s), one or more lipidation(s), such as a farnesylation or a palmitoylation, a structural rearrangement such as disulfide bridge formation and/or such as cleavage within the peptide sequence.

An analog of an amino acid sequence has, moreover, substantially the same biological activity as this amino acid sequence.

It is, moreover, known that a primary amino acid sequence can comprise sites specifically recognized by protease-type enzymes, such as trypsin, which, once these sites have actually been recognized, will induce the cleavage of the sequence by proteolysis. This proteolysis results in the production of various peptides, or fragments of amino acid sequences of the invention.

Consequently, the invention also extends to the dermicidin fragments resulting, where appropriate, from its proteolysis.

For the purposes of the invention, the expression “fragment of an amino acid sequence” means any portion of the amino acid sequence in accordance with the invention comprising from 3 to 48 consecutive amino acids of said sequence, preferably from 6 to 36, preferably from 8 to 32 and more preferentially from 10 to 30 consecutive amino acids of said sequence, and having a biological activity of the same nature.

According to one embodiment, an amino acid sequence suitable for the invention can be an amino acid sequence represented by a sequence chosen from SEQ ID No.: 11 to 20, in particular from SEQ ID No.: 11, SEQ ID No.: 12, SEQ ID No.: 13, or an analog or fragment thereof.

According to one preferred embodiment, an amino acid sequence suitable for the invention can be an amino acid sequence represented by a sequence chosen from SEQ ID No.: 14, SEQ ID No.: 15, SEQ ID No.: 16, SEQ ID No.: 17, SEQ ID No.: 18, SEQ ID No.: 19, SEQ ID No.: 20, or an analog or fragment thereof.

More preferably, an amino acid sequence suitable for the invention can be an amino acid sequence represented by a sequence chosen from SEQ ID No.: 16, SEQ ID No.: 17 or SEQ ID No.: 18, SEQ ID No.: 19 or SEQ ID No.: 20, or an analog or fragment thereof.

Preferably, an amino acid sequence suitable for the invention can be an amino acid sequence represented by a sequence chosen from SEQ ID No.: 17, SEQ ID No.: 18, SEQ ID No.: 19 or SEQ ID No.: 20, or an analog or fragment thereof.

Even more preferably, an amino acid sequence suitable for the invention can be an amino acid sequence represented by a sequence chosen from SEQ ID No.: 17, SEQ ID No.: 18, or an analog or fragment thereof.

According to another embodiment, a polypeptide suitable for the invention can also be a natural or synthetic amino acid sequence, where appropriate which can be obtained after enzymatic or chemical lysis of dermicidin or by chemical or biological synthesis or by extraction from a biological tissue, for instance the skin, naturally expressing this amino acid sequence or after transfection thereof, and also the various post-translational forms thereof, or else any natural or synthetic amino acid sequence, the sequence of which totally or partially comprises an abovementioned amino acid sequence, for example the variants and the analogs.

Those skilled in the art can obtain an amino acid sequence in accordance with the invention by means of methods based on recombinant DNA, for instance those described in the manual <<Molecular Cloning—A Laboratory Manual>> (2nd edition), Sambrook et al., 1989, Vol. I-III, Coldspring Harbor Laboratory, Coldspring Harbor Press, NY, (Sambrook).

According to one subject, the present invention relates as such to an isolated peptide represented by an amino acid sequence chosen from SEQ ID No.: 17 or SEQ ID No.: 18, or an analog or fragment thereof.

According to another embodiment, an amino acid sequence suitable for the invention can also be an amino acid sequence as previously defined, fused with another amino acid sequence, a hydrophilic or hydrophobic targeting agent, a bioconversion precursor, or a luminescent, radioactive or colorimetric labeling agent.

In a nonlimiting manner, mention may be made, as examples of compounds which can be coupled to an amino acid sequence in accordance with the invention, of fluorescent proteins such as Green Fluorescent Protein, fluorescent chemical compounds, such as rhodamine, fluorescein, or Texas Red®, phosphorescent compounds, radioactive elements, such as ³H, ¹⁴C, ³⁵S, ¹²¹I or ¹²⁵I, or colorimetric labeling agents such as chromogenic substrates sensitive to the action of galactosidase, of peroxidase, of chloramphenicol acetyltransferase, of luciferase or of alkaline phosphatase.

Depending on the nature of the compounds which can be coupled with an amino acid sequence of the invention, the coupling can be carried out by chemical methods, in particular by means of reactive chemical functions, or by molecular biology methods known to those skilled in the art.

Advantageously, an amino acid sequence of the invention can be encoded by a nucleic acid sequence chosen from a sequence represented by SEQ ID No.: 1, SEQ ID No.: 2, SEQ ID No.: 3, SEQ ID No.: 4, SEQ ID No.: 5, SEQ ID No.: 6, SEQ ID No.: 7, SEQ ID No.: 8, SEQ ID No.: 9, SEQ ID No.: 10, or an analog or fragment thereof.

According to one embodiment, the present invention also relates to nucleic acid sequences encoding an amino acid sequence of the invention and the employment thereof in the various uses and methods in accordance with the invention.

Thus, the present invention also relates to a nucleic acid sequence, in particular a deoxyribonucleic acid sequence or a ribonucleic acid sequence, represented by SEQ ID No.: 1, or an analog or fragment thereof.

For the purposes of the present invention, the expression “fragment of a nucleic acid sequence” means a nucleic acid sequence comprising from 9 to 144 consecutive base pairs of said sequence, preferably from 18 to 108, preferably from 24 to 96 and more preferentially from 30 to 90 consecutive base pairs of said sequence, and encoding an amino acid sequence having a biological activity of the same nature as the amino acid sequence encoded by said sequence.

For the purposes of the present invention, the expression “analog of a nucleic acid sequence” means a nucleic acid sequence having a sequence identity of at least 85%, preferably of at least 90% and more preferentially of at least 95% with said sequence, and encoding an amino acid sequence having a biological activity of the same nature as the amino acid sequence encoded by said sequence.

The expression “analog of a nucleic acid sequence” is intended to denote a nucleic acid sequence optionally resulting from the degeneracy of the nucleic acid code, and encoding an amino acid sequence in accordance with the invention, in particular as previously defined.

According to one preferred embodiment, a nucleic acid sequence of the invention can be represented by a sequence chosen from SEQ ID No.: 2, SEQ ID No.: 3, SEQ ID No.: 4, SEQ ID No.: 5, SEQ ID No.: 6, SEQ ID No.: 7, SEQ ID No.: 8, SEQ ID No.: 9 and SEQ ID No.: 10, or an analog or fragment thereof.

A nucleic acid sequence of the invention can be of any possible origin, namely animal, in particular mammalian and even more particularly human, or plant, or from microorganisms, such as, for example, viruses, phages or bacteria, inter alia, or else from fungi, without any preconception as to whether or not they are naturally present in said organism of origin.

According to one embodiment, the invention also relates to the isolated and purified nucleic acid sequences encoding an amino acid sequence under consideration according to the invention, and also to the analogs and fragments thereof.

A nucleic acid sequence in accordance with the invention can comprise a sense, antisense or interfering sequence corresponding to a sequence encoding a polypeptide in accordance with the invention.

A subject of the invention is also nucleic acid sequences, in particular ribonucleic acid sequences or deoxyribonucleic acid sequences, comprising a sense or antisense, in particular small interfering RNA (siRNA), sequence corresponding at least to a sequence encoding a polypeptide of the invention or a nucleic acid sequence represented by SEQ ID No.: 1, SEQ ID No.: 2, SEQ ID No.: 3, SEQ ID No.: 4, SEQ ID No.: 5, SEQ ID No.: 6, SEQ ID No.: 7, SEQ ID No.: 8, SEQ ID No.: 9, SEQ ID No.: 10, or an analog or fragment thereof.

Aged Skin and Signs of Skin Aging

The term “aged skin” means a general state of the skin resulting from chronological aging and/or photoinduced aging.

The expression “signs of skin aging” means any of the modifications of the external appearance of the skin due to aging, whether it is of chronological and/or photo-induced origin.

By way of example of these modifications considered in the invention, mention may be made of wrinkles and fine lines, withered skin, a lack of elasticity and/or of tonicity of the skin, thinning of the dermis and/or degradation of the collagen fibers, which leads to the appearance of flaccid and wrinkled skin.

Said expression also means all the internal modifications of the skin which are not systematically reflected by a modified external appearance, for instance all the internal degradations of the skin, and more particularly the degradation of the elastin fibers, or elastic fibers, subsequent to exposure to ultraviolet radiation.

In particular, the signs of skin aging that are targeted by the invention are chosen from thinning of the skin, a loss of firmness, a loss of elasticity, a loss of density or a loss of tonicity of the skin, dryness of the skin, the appearance of a marked microrelief of the skin, the formation and/or the presence of fine lines and/or of wrinkles, a modification of the radiance of the skin complexion, a wizened appearance of the skin, a modification of the odor of the skin, sagging of the skin and withering of the skin.

Preferably, the signs of skin aging that are targeted by the invention are chosen from thinning of the skin, the appearance of a marked microrelief of the skin, the formation and/or the presence of fine lines and/or of wrinkles, sagging of the skin and withering of the skin.

More preferably, the signs of skin aging that are targeted by the invention are chosen from the appearance of a marked microrelief of the skin, the formation and/or the presence of fine lines and/or of wrinkles, sagging of the skin and withering of the skin.

The term “skin moisturization” means all of the cellular and molecular mechanisms which result in providing and maintaining the presence of an amount of physiological saline in the epidermis and the dermis, and also the resulting amount of water.

According to one aspect, the invention aims to maintain or even stimulate the homeostasis of these mechanisms, and thus to promote and/or reinforce skin moisturization. Thus, according to one aspect, the invention applies to skin which has a physiological moisturized state, also described as normal.

According to another aspect, the invention aims to restore the balance or reduce the risk of occurrence of an imbalance in the homeostasis of these mechanisms, and thus to prevent and/or treat dry skin and/or the signs of dryness of the skin.

The expression “signs of dryness of the skin” means all the modifications of the appearance of the skin due to a shortage of water in the epidermis and/or the dermis.

Depending on the degree of manifestation of the shortage of water in the skin, dry skin can appear coarse to the touch, wrinkled, or even covered with squamae. Dry skin can be essentially manifested by a sensation of tautness and/or tension.

The term “dry skin” means a general state of the skin resulting from a shortage of water in the epidermis and/or the dermis.

Dry skin can be manifested by a desquamation problem and exhibit various stages depending on the severity of this desquamation.

When the skin is slightly dry, these squamae are abundant but barely visible to the naked eye; elimination is carried out corneocyte by corneocyte. These squamae are increasingly few in number, but increasingly visible to the naked eye when this disorder worsens; the masses can comprise several hundred corneocytes, thus representing more or less large masses, called squamae.

Dryness of the skin may be constitutional or acquired.

In the case of constitutional dry skin, two categories may be distinguished: pathological skin and nonpathological skin.

Pathological constitutional dry skin is essentially represented by atopic dermatitis and ichthyoses. It is virtually independent of the external conditions and the cause is known or unknown genetic modifications. Among the known genetic modifications affecting skin moisturization, mention may be made, for example, of modifications of the transglutaminase-1 gene or those of the filaggrin gene.

According to one embodiment, also described is a pharmaceutical or dermatological composition comprising, in a physiologically acceptable medium, an effective amount (i) of at least one amino acid sequence of the invention, or (ii) of at least one nucleic acid sequence of the invention, or (iii) of at least one agent for modulating the activity, the expression or the maturation of said amino acid sequence or of said nucleic acid sequence, as an active agent for preventing and/or treating pathological constitutional dry skin chosen from atopic dermatitis and ichthyoses.

In the case of nonpathological constitutional dry skin, the severity of the state of dryness may depend, for its part, on external factors. Included in this skin category are senile skin (characterized by a general decrease in skin metabolism with age), fragile skin (very sensitive to external factors and often accompanied by erythema and rosacea) and common xerosis (of probable genetic origin and manifesting mainly on the face, the limbs and the back of the hands).

In the case of acquired dry skin, the intervention of external parameters such as exposure to chemical agents, inclement climatic conditions, sunlight or certain therapeutic treatments (for example retinoids) is a determining factor. Under these external influences, the epidermis may then become momentarily and locally dry. This may concern any type of epidermis.

Irrespective of its origin, skin suffering from dryness may generally present the following signs: appearance which is coarse to the touch and scaly, and decreased suppleness and elasticity.

Dry skin, also known as “xerosis”, may appear at any age, and may be unrelated to a pathological condition. In this case, it will be referred to as “acquired” dryness.

However, xerosis becomes more frequent and troublesome with age, especially for women. It is then referred to as senile xerosis. Moreover, women generally suffer a worsening of dryness of the skin during the menopause, probably due to the characteristic hormonal imbalance of this phenomenon. The areas most affected are the lower part of the legs, the back of the forearms and the hands.

As previously mentioned, acquired dryness can be influenced by external factors. For example, the appearance of dry skin can be promoted by cold, dry and winter weather. It is then referred to as winter xerosis. Dryness of the skin may also be induced by an exogenous stress, of chemical origin, for example of anionic detergent type, or of mechanical origin (rubbing or shaving).

According to one embodiment, a cosmetic use of the invention may advantageously be suitable for preventing and/or treating senile or fragile dry skin, or xerosis, in particular chosen from common xerosis, senile xerosis and winter xerosis.

Although no study has demonstrated any effect of dryness on the origin and formation of the wrinkles and fine lines which are essentially attributable to aging, from the visual point of view, dry skin makes them more obvious. Dry skin can therefore be associated with aged skin exhibiting wrinkles or fine lines.

Moreover, from a sensory point of view, dryness of the skin is characterized by a feeling of tautness and/or itching. For obvious reasons, these manifestations are not only a source of discomfort, or even pain, but also have an unattractive appearance.

According to one embodiment, also described is a cosmetic use which can advantageously be suitable for preventing and/or treating feelings of tautness and/or itching associated with dry skin.

By way of example of signs of dryness of the skin which are considered in the invention, mention may be made of withered skin, a lack of elasticity, suppleness and/or tonicity of the skin, a coarse feel, the presence of cracks, desquamation, the presence of scales, or wrinkles and fine lines associated with dry skin.

The signs of dryness of the skin which are considered are also all the internal modifications of the skin which are not systematically reflected by a modified external appearance, for instance all the internal degradations of the skin, and more particularly the degradation of the elastin fibers, or elastic fibers, subsequent to drying of the dermis. According to one preferred aspect of the invention, the signs of dryness of the skin are chosen from withered skin, a lack of elasticity, of suppleness and/or of tonicity of the skin, a coarse feel, the presence of cracks, a desquamation, the presence of scales, wrinkles and fine lines associated with dry skin, and a feeling of tautness and/or itching.

According to one very preferred aspect of the invention, the aged skin or the signs of skin aging may or may not be associated with dryness of the skin.

Biomarker

The present invention relates to the use of at least one amino acid sequence of the invention, or of at least one nucleic acid sequence of the invention, as a biomarker for a state of the skin.

Also described is the use of at least one amino acid sequence of the invention, or of at least one nucleic acid sequence of the invention, as a biomarker for a moisturization state of the skin, and more particularly dry skin and/or the signs of dryness of the skin.

Preferably, a use in accordance with the invention makes it possible to characterize a state of the skin, such as aged skin and/or the signs of skin aging, which may or may not be associated with dryness of the skin.

According to one embodiment, a decrease in the activity, in the expression or in the maturation of said biomarker may be indicative of aged skin and/or of signs of skin aging, which may or may not be associated with dryness of the skin.

According to one embodiment, it is also indicated that a decrease in the activity, in the expression or in the maturation of said biomarker may be indicative of skin with a lack of moisturization, and more particularly of dry skin and/or of signs of dryness of the skin. According to another of its aspects, the present invention relates to the use of at least one amino acid sequence of the invention, or at least one nucleic acid sequence of the invention, for characterizing the efficacy of a cosmetic treatment for the skin, and preferably for aged skin and/or for the signs of skin aging, which may or may not be associated with dryness of the skin.

Also described is the use of at least one amino acid sequence of the invention, or of at least one nucleic acid sequence of the invention, for characterizing the efficacy of a cosmetic treatment for dry skin and/or for the signs of dryness of the skin.

According to one embodiment, an increase in the activity, in the expression or in the maturation of said biomarker may be indicative of an effective cosmetic treatment for exerting a beneficial effect on the skin and more preferentially an effect with regard to aged skin and/or to signs of skin aging, which may or may not be associated with dryness of the skin.

It is also indicated that an increase in the activity, in the expression or in the maturation of said biomarker may be indicative of an effective cosmetic treatment for exerting a beneficial effect on skin with a lack of moisturization, and more preferentially an effect with regard to dry skin and/or to signs of dryness of the skin.

In one use in accordance with the invention, a decrease or increase in the activity, in the expression or in the maturation of said biomarker can be determined by comparison with a reference measurement obtained according to any method known to those skilled in the art.

A “reference measurement” from the viewpoint of a given parameter is a qualitative or quantitative measurement of this parameter carried out under “control” or “normal” conditions, for example determined in a reference sample, or determined in a sample in the absence of a treatment presumed to have an effect on the parameter.

For example, a reference measurement for an amino acid sequence or a nucleic acid sequence in accordance with the invention can be a quantitative or qualitative value relative to the expression, the maturation or the activity of said sequences, determined in a sample of young and physiologically healthy skin, or determined in a sample of skin, in particular of aged skin, before a cosmetic treatment.

Likewise for example, a reference measurement for an amino acid sequence or a nucleic acid sequence in accordance with the invention can be a quantitative or qualitative value relative to the expression, the maturation or the activity of said sequences, determined in a sample of physiologically healthy and normally moisturized skin, or determined in a sample of dry skin, before a cosmetic treatment.

Preferably, a reference measurement is a statistical measurement, i.e. a measurement having been repeated on various samples so as to obtain a mean.

The reference measurement can be carried out in parallel with or sequentially to the test measurement.

It can also be a “historical” measurement, i.e. one carried out prior to the test measurement, and stored, for example in a database, for the purpose of subsequent use.

A comparison of the test measurement to a reference measurement, and observation of the deviation or an absence of deviation between the two measurements, makes it possible to extract information regarding the parameter measured, for example the decrease or increase in the expression, in the maturation or in the activity of an amino acid sequence or of a nucleic acid sequence in accordance with the invention.

Such information can be subsequently used to determine the young or aged nature of a skin. Likewise, such information can be subsequently used to determine the normally moisturized or dry nature of a skin.

A method according to the invention can also be carried out on a sample of skin, taken from an epidermal cell model, or from a reconstructed isolated skin in order to describe the state thereof.

According to one embodiment, the invention relates to a method, in particular an in vitro or ex vivo method, for characterizing a state of the skin.

Also described is a method, in particular an in vitro or ex vivo method, for characterizing a moisturization state of the skin.

A method of the invention advantageously makes it possible to characterize an aged state of the skin and/or signs of skin aging, and more particularly to characterize an aged state of the skin of chronological and/or photoinduced origin.

It is also indicated that a method of the invention advantageously makes it possible to characterize a dry state of the skin and/or signs of dryness of the skin.

According to another embodiment, the invention relates to a cosmetic, or nontherapeutic, method, in particular an in vitro or ex vivo method, for characterizing the efficacy of a cosmetic treatment of the skin, and preferably of aged skin and/or of the signs of skin aging, which may or may not be associated with dryness of the skin, in an individual in need thereof, comprising at least the steps consisting in:

a) carrying out, before the implementation of the cosmetic treatment, in a first isolated skin sample taken from said individual, at least one first qualitative or quantitative measurement of the expression, of the maturation or of the activity of at least one amino acid sequence of the invention or of at least one nucleic acid sequence of the invention,

b) carrying out, after the implementation of the cosmetic treatment, in a second isolated skin sample taken from said individual, at least one second qualitative or quantitative measurement of the expression, of the maturation or of the activity of said amino acid sequence of the invention or of said nucleic acid sequence of the invention, and

c) comparing the first and second measurements, in particular in order to deduce therefrom information relating to at least one effect of the implementation of the cosmetic treatment.

It goes without saying that the measurements carried out in steps a) and b) must be comparable to one another, and therefore relate to the same parameter.

Preferably, a method of the invention makes it possible to demonstrate an effect of a cosmetic treatment capable of causing the regression of aged skin and/or the signs of skin aging, which may or may not be associated with dryness of the skin.

Also described is a cosmetic, or nontherapeutic, method, in particular an in vitro or ex vivo method, for characterizing the efficacy of a cosmetic treatment for dry skin and/or for the signs of dryness of the skin, in an individual in need thereof, comprising at least the steps consisting in:

a) carrying out, before the implementation of the cosmetic treatment, in a first isolated skin sample taken from said individual, at least one first qualitative or quantitative measurement of the expression, of the maturation or of the activity of an amino acid sequence of the invention or of a nucleic acid sequence of the invention,

b) carrying out, after the implementation of the cosmetic treatment, in a second isolated skin sample taken from said individual, at least one second qualitative or quantitative measurement of the expression, of the maturation or of the activity of said amino acid sequence of the invention or of said nucleic acid sequence of the invention, and

c) comparing the first and second measurements, in particular in order to deduce therefrom information relating to at least one effect of the implementation of the cosmetic treatment.

It goes without saying that the measurements carried out in steps a) and b) must be comparable to one another, and therefore relate to the same parameter.

Preferably, it is also indicated that a method of the invention makes it possible to demonstrate an effect of a cosmetic treatment capable of causing the regression of dry skin and/or the signs of dryness of the skin.

The qualitative or quantitative measurement of the expression, of the maturation or of the activity of a nucleic acid sequence of the invention can be determined by any method known to those skilled in the art.

By way of example of methods suitable for the invention, mention may be made of the quantitative polymerase chain reaction (Q-PCR) or nonquantitative polymerase chain reaction (PCR), in the presence or absence of reverse transcriptase (RT-PCR or Q-RT-PCR), Northern blotting, the ribonuclease protection assay method, methods with DNA chips, methods with transcriptome chips, methods with oligonucleotide chips, and in situ hybridization methods.

By way of example of agents suitable for detecting a nucleic acid sequence of the invention, and in particular an mRNA sequence, mention may be made of labeled nucleic acid probes which can hybridize to a nucleic acid sequence of the invention.

Such a nucleic acid probe can be easily obtained by any method known to those skilled in the art.

Thus, the nucleic acid sequences in accordance with the invention can be used to prepare sense and/or antisense oligonucleotide primers which hybridize, under high stringency conditions, to at least one of the sequences SEQ ID No.: 1, SEQ ID No.: 2, SEQ ID No.: 3, SEQ ID No.: 4, SEQ ID No.: 5, SEQ ID No.: 6, SEQ ID No.: 7, SEQ ID No.: 8, SEQ ID No.: 9, SEQ ID No.: 10, or an analog or fragment thereof.

The expression of a nucleic acid sequence can also be determined, indirectly, by determining the expression of the amino acid sequence encoded by said sequence, by means of any technique known in the field, such as Western blotting, ELISA, the BRADFORD method or the LOWRY method, or as indicated hereinafter.

The qualitative or quantitative measurement of the expression, of the maturation or of the activity of an amino acid sequence of the invention can be carried out by means of any method known to those skilled in the art.

By way of methods for detecting the expression, the maturation or the activity of an amino acid sequence, mention may be made of Western blotting, slot blotting, dot blotting, ELISA (Enzyme Linked Immuno-Sorbent Assay) methods of singleplex or multiplex type, proteomic or glycomic methods, methods for staining polypeptides in a polyacrylamide gel with a silver-based stain, with Coomassie blue or with SYPRO, immunofluorescence methods, UV absorption methods, immunohistochemical methods by conventional, electron or confocal microscopy, FRET (fluorescence resonance energy transfer) methods, TR-FRET (time resolved FRET) methods, FLIM (fluorescence lifetime imaging microscopy) methods, FSPIM (fluorescence spectral imaging microscopy) methods, FRAP (fluorescence recovery after photobleaching) methods, reporter gene methods, AFM (atomic force microscopy) methods, surface plasmon resonance methods, microcalorimetry methods, flow cytometry methods, biosensor methods, radioimmunoassay (RIA) methods, isoelectric focusing methods, and enzymatic tests, methods using peptide chips, sugar chips, antibody chips, mass spectrometry methods, and spectrometry methods of SELDI-TOF type (Ciphergen).

More generally, immunoenzymatic assay methods using protein solutions, which are more quantitative and sensitive, can in particular be used. These ELISA-type methods combine pairings of target-antigen-specific capture antibody and detection antibody. Commercial antibodies or specifically developed polyclonal, monoclonal or recombinant antibodies can be used. High capacity multiplex ELISA techniques can also be used. Mention may thus be made of the multiplex approach such as antibodies on Luminex beads (for example, Bioplex from Bio-Rad) and such as antibodies on a flat surface (antibody arrays) (for example, the approach proposed by the company MesoScale Discovery).

In particular, it may be advantageous to detect the expression of an amino acid sequence of the invention by means of an antibody, where appropriate in labeled form. Such an antibody can be labeled by means of a substance that is directly detectable or detectable by reaction with another reagent.

The term “antibody” is intended to denote, generally, monoclonal or polyclonal antibodies, and also immunoglobulin fragments capable of binding an antigen and which can be produced by any genetic engineering technique known to those skilled in the art or by enzymatic or chemical cleavage of an intact antibody.

An antibody which can be used as a tool for evaluating a state of an epidermis can be obtained by any method known to those skilled in the art, as described in “Antibodies: A Laboratory Manual”, Cold Spring Harbor Laboratory Press, Cold Spring Harbor, N.Y. (1990).

According to one preferred embodiment, it may be also advantageous to detect the expression of an amino acid sequence of the invention by means of an “iTRAQ” differential proteomic method.

According to one preferred embodiment, it may also be advantageous to detect the expression of an amino acid sequence of the invention by means of Western blotting.

Such a method is known to those skilled in the art and can advantageously be carried out as described in the examples hereinafter.

In particular, the term “activity” with regard to an amino acid sequence of the invention means an antimicrobial activity, a cell survival stimulating activity, a cell proliferation stimulating activity, or an activity for reducing or preventing aged skin and/or the signs of skin aging, which may or may not be associated with dryness of the skin, as indicated above.

Likewise in particular, the term “activity” with regard to an amino acid sequence of the invention means an antimicrobial activity, a cell survival stimulating activity, a cell proliferation stimulating activity, or an activity for reducing or preventing dry skin and/or the signs of dryness of the skin, as indicated above.

Such an activity can be determined by any method known to those skilled in the art, for instance by evaluating the proliferation or the survival of epidermal cells in culture, such as keratinocytes, or by evaluating the antimicrobial activity on bacteria in culture, such as Staphylococcus aureus or Escherichia coli.

Preferably, the determination of a state of the skin or the characterization of the efficacy of a cosmetic treatment for the skin can be carried out by measuring the variation in the expression of an amino acid sequence of the invention, and which is preferably represented by a sequence chosen from SEQ ID No.: 16, SEQ ID No.: 17, SEQ ID No.: 18, SEQ ID No.: 19 or SEQ ID No.: 20, or an analog or fragment thereof.

The methods of the invention are particularly advantageous since the implementation thereof does not require recourse to an invasive technique. A sample of epidermis can thus be obtained by “stripping” techniques and directly analyzed by a conventional analysis technique known to those skilled in the art.

These strippings are adhesive surfaces applied to the surface of the epidermis, such as Blenderm® from 3M, D′ squam (commercial adhesive from CuDERM), cyanoacrylate glue or the varnish “stripping” method. By virtue of these strippings, the adherent corneocytes and the content of their intercellular spaces can be sampled and subsequently subjected to extraction in order to obtain the protein content.

The taking of a sample suitable for a method of the invention can also be carried out more directly by “washing” the skin surface, by means, for example, of accessories of the vane turbine type or of the spiral cell type as described in the patent FR 2 667 778 combined with a fluid circuit, or simply by addition/sampling of a drop of buffer at the surface of the skin.

By way of indication, other sampling methods suitable for implementing the invention may be mentioned, such as methods by scraping the upper part of the stratum corneum by means of a twin blade system. This technique makes it possible to collect squamae which can then be directly analyzed by various techniques in order to determine the mineral, amino acid or lipid contents.

Advantageously, one of the markers of the invention can be used for the purposes of more efficient and more rigourous preclinical selection, of individuals, with a view to evaluating the efficacy of treatment or of a cosmetic active agent for skincare.

Likewise advantageously, one of the markers of the invention can be used for the purposes of more efficient and more rigourous preclinical selection, of individuals, with a view to evaluating the efficacy of treatment or of a cosmetic active agent for the care of dry skin.

Likewise, a biomarker of the invention can advantageously be used as previously indicated for evaluating the efficacy of an active agent, in vitro, ex vivo or in vivo, with regard to a lack of moisturization of the skin, or dry skin or the signs of dryness of the skin.

Likewise, a biomarker of the invention can advantageously be used as previously indicated for evaluating the efficacy of an active agent, in vitro, ex vivo or in vivo.

Likewise, a biomarker of the invention can be used for establishing personalized advice for a cosmetic treatment for an individual according to the latter's skin biomarker expression profile.

Screening

According to one of its aspects, the present invention relates to the use of an amino acid or nucleic acid sequence of the invention, for screening for or in a method for screening for, in particular in vitro or ex vivo, active agents or physical treatments which are particularly suitable for skincare. The active agents or the physical treatments screened can in particular be suitable for the care of aged skin, and/or for the prevention and/or treatment of the signs of skin aging, which may or may not be associated with dryness of the skin.

Also described is the use of an amino acid or nucleic acid sequence of the invention, for screening for or in a method for screening for, in particular in vitro or ex vivo, active agents or physical treatments which are particularly suitable for skincare and intended for promoting and/or reinforcing skin moisturization.

Likewise, the active agents or the physical treatments screened can in particular be suitable for the care of dry skin, and/or for preventing and/or treating the signs of dryness of the skin.

A use or a method of the invention can comprise the comparison of a measurement of the activity, of the expression or of the maturation of an amino acid sequence or of a nucleic acid sequence in accordance with the invention, to a reference measurement.

A reference measurement can be as previously defined.

In particular, a reference measurement can be a quantitative or qualitative value relating to the expression, the maturation or the activity of said sequences, determined in a sample in the absence of active agent or of physical treatment tested.

Thus, a reference measurement can be obtained by repeating the steps of a method of the invention, in particular steps a), b) and c) of a method of the invention as previously defined, in the absence of biological or chemical compounds, or physical treatments to be tested.

A comparison of the test measurement to a reference measurement, and observation of a deviation or of an absence of deviation between the two measurements, makes it possible to extract information with regard to the effect of the active agent or of the physical treatment tested.

The qualitative or quantitative determination of the expression, of the maturation or of the activity of an amino acid sequence or of a nucleic acid sequence of the invention can be carried out by any method known to those skilled in the art, and in particular as previously described.

According to one embodiment, the screening for an active agent or a physical treatment capable of modulating the activity of an amino acid sequence of the invention can be carried out by measuring the activity or the expression of a target molecule belonging to the signaling or metabolic pathways in which said amino acid sequence may be involved, such as, for example, a reporter gene system.

According to one embodiment, a method of the invention can be carried out in an acellular system, i.e. in a system which does not comprise cells but which reproduces cell functions, or in an isolated cell sample.

A method in accordance with the invention can be carried out on an isolated cell sample, an acellular sample, on an isolated amino acid sequence or on an isolated nucleic acid sequence of the invention, obtained by skin biopsy, from cells in culture, in particular from an epidermal model, or from a noninvasive skin surface specimen, in particular taken by tape-stripping of the stratum corneum, or by simple washing, as previously described.

Advantageously, by way of a cell sample suitable for the invention, mention may be made of a sample of keratinocytes or any other skin cell type expressing an amino acid sequence of the invention.

Preferably, the screening for an active agent or for a physical treatment can be carried out by measuring the variation in the expression, in the presence and in the absence of the active agent or of the physical treatment screened, of an amino acid sequence of the invention, and which is preferably represented by a sequence chosen from SEQ ID No.: 16, SEQ ID No.: 17 or SEQ ID No.: 18, or an analog or fragment thereof.

Modulating Agent

For the purposes of the present invention, the expression “modulating agent” or “active agent or a physical treatment capable of modulating the expression, the maturation or the activity of an amino acid sequence or of a nucleic acid sequence in accordance with the invention” means any compound or physical phenomenon capable of acting, directly or indirectly, on at least one amino acid sequence or one nucleic acid sequence in accordance with the invention, or on an element of an intracellular or extracellular signaling pathway, or of a metabolic pathway, or for regulating transcription and/or translation, involving said amino acid sequence or said nucleic acid sequence.

For the purposes of the invention, the term “modulating” means, from the viewpoint of a given effect, the action of stimulating or inhibiting this effect.

The active agents or the physical treatments resulting from a screening according to the invention can be advantageously used for cosmetic purposes, in particular from the viewpoint of aged skin or the signs of skin aging, which may or may not be associated with dryness of the skin. It is also indicated that the active agents or the physical treatments resulting from a screening according to the invention can be advantageously used for cosmetic purposes, in particular from the viewpoint of skin moisturization, and in particular of dry skin or the signs of dryness of the skin.

According to one embodiment, an active agent of the invention can be an agent for stimulating the activity, the expression or the maturation of an amino acid sequence of the invention.

In particular, a stimulating agent can be chosen from a Bifidobacterium sp. lysate and a mixture composed of a Bifidobacterium sp. lysate and of phytosphingosine salicylate.

Preferably, a Bifidobacterium sp. lysate can be a Bifidobacterium longum lysate. A Bifidobacterium longum lysate suitable for the invention can be the lysate registered under the INCI name: Bifidat ferment Lysate, under the EINECS name: Bifidobacterium longum, under the EINECS No.: 306-168-4 and under the CAS No.: 96507-89-0. Such a lysate is in particular sold under the name Repair Complex CLR® by the company K. Richter GmbH.

A mixture composed of a Bifidobacterium sp. lysate and of phytosphingosine salicylate according to the invention can comprise a Bifidobacterium longum lysate as previously defined and a phytosphingosine salicylate derivative.

A phytosphingosine salicylate derivative suitable for the invention can be the derivative sold by the company Evonick Goldschmidt under the name Phytosphingosine SLC®.

A mixture according to the invention can comprise a Bifidobacterium sp. lysate in a proportion of 10% and a phytosphingosine salicylate derivative in a proportion of 0.002%.

By way of modulating agent capable of being screened according to a use or a method in accordance with the invention, mention may also be made of antibodies or interfering RNAs.

Compositions

The present invention also relates to compositions, in particular cosmetic compositions, comprising, in a physiologically or cosmetically acceptable medium, an effective amount of at least one amino acid sequence of the invention, or of at least one nucleic acid sequence of the invention, or of at least one active agent capable of modulating the activity, the expression or the maturation of said amino acid sequence or of said nucleic acid sequence.

More particularly, the invention relates to a cosmetic composition comprising a peptide represented by an amino acid sequence chosen from SEQ ID No.: 17 or SEQ ID No.: 18, or an analog or fragment thereof, or a nucleic acid sequence encoding such a peptide.

For the purposes of the present invention, the expression “physiologically acceptable medium” is intended to denote a medium suitable for the administration of a composition topically to the skin, the scalp or the lips, or orally or parenterally, such as intradermally or subcutaneously.

A composition of the invention may contain adjuvants that are customary in the field under consideration, such as hydrophilic or lipophilic gelling agents, hydrophilic or lipophilic additives, preservatives, antioxidants, solvents, fragrances, fillers, screening agents, odor absorbers and colorants.

The amounts of the various constituents of the compositions according to the invention are those conventionally used in the fields under consideration.

The amount of amino acid sequence or nucleic acid sequence of the invention or of active agent in accordance with the invention, contained in a composition of the invention, also referred to as “effective amount”, depends of course on the nature of the active agent and on the desired effect and can therefore vary to a large extent.

To give an order of magnitude, a composition can contain an amino acid sequence or a nucleic acid sequence or an active agent in accordance with the invention in an amount representing from 0.00001% to 50% of the total weight of the composition, in particular in an amount representing from 0.001% to 10% of the total weight of the composition and more particularly in an amount representing from 0.1% to 1% of the total weight of the composition.

According to another embodiment, a cosmetic composition of the invention may also comprise at least one additional cosmetic and/or therapeutic active agent.

Additional Active Agents

As examples of additional active agents that can be used in the context of the present invention, mention may be made of cosmetic oils, such as silicone oils, plant oils of triglyceride type, hydrocarbon-based oils, such as parleam oil, and esters of fatty acids and of fatty alcohols.

It may also be possible to use other active agents for improving the state of the skin and/or of its appendages, such as moisturizing or humidifying active agents or active agents for improving the natural lipid barrier, such as ceramides, cholesterol sulfates and/or fatty acids, and mixtures thereof.

It may also be possible to use other active agents for improving the state of the skin and/or its appendages, such as anti-aging active agents.

It may also be possible to use enzymes which have an activity on the skin and/or its appendages, such as proteases, lipases, glucosidases, amidases, cerebrosidases and/or melanases, and mixtures thereof.

Other examples of active agents suitable for the implementation of the present invention are: analgesic active agents, anti-yeast active agents, antibacterial active agents, antiparasitic active agents, antifungal active agents, anti-viral active agents, steroidal anti-inflammatory active agents, anesthetic active agents, antipruritic active agents, keratolytic active agents, free-radical scavenging active agents, antiseborrheic active agents, antidandruff active agents, anti-acne active agents, active agents aimed at preventing aging of the skin and/or improving the state thereof, antidermatitis active agents, anti-irritant active agents, immunomodulator active agents, active agents for treating dry skin, antiperspirant active agents, antipsoriatic active agents, UV-protection active agents, antihistamine active agents, healing active agents, self-tanning active agents, antioxidants such as green tea or active fragments thereof, glycerol, laponite, caffeine, aromatic essential oils, dyes, depigmenting active agents, liporegulators, softening, refreshing, deodorizing, desensitizing, bleaching or nourishing active agents, active agents for reducing skin differentiation and/or proliferation and/or pigmentation, and mixtures thereof.

By way of additional active agents which may be more particularly suitable for the invention, mention may also be made of probiotic microorganisms, other than those considered in the lysate previously described, prebiotic active agents, active agents for promoting the synthesis of skin defense factor, active agents for re-establishing the differentiation/proliferation equilibrium of epidermal cells, in particular such as active agents of retinol or retinol-like type, moisturizing active agents or active agents for stimulating skin protease activity, in particular cathepsin D activity.

Cosmetic Use

The present invention relates to the cosmetic use of an effective amount of at least one amino acid or nucleic acid sequence of the invention, or of at least one agent for modulating the activity, the expression or the maturation of said amino acid sequence or of said nucleic acid sequence, and in particular a modulating agent as previously defined, as an active agent for preventing and/or treating aged skin and/or the signs of skin aging, which may or may not be associated with dryness of the skin.

Also described is the cosmetic use of an effective amount of at least one amino acid or nucleic acid sequence of the invention, or of at least one agent for modulating the activity, the expression or the maturation of said amino acid sequence or of said nucleic acid sequence, and in particular a modulating agent as previously defined, as an active agent for promoting and/or reinforcing skin moisturization.

It is also indicated that a use of the invention can be devoted to preventing and/or treating dry skin and/or signs of dryness of the skin.

According to another aspect, the present invention relates to a cosmetic, or nontherapeutic, method for preventing and/or treating aged skin and/or signs of skin aging, which may or may not be associated with dryness of the skin, in an individual in need thereof, the method comprising at least one step consisting in administering, to said individual, at least one composition comprising, as active agent, (i) at least one amino acid sequence of the invention or (ii) at least one nucleic acid sequence of the invention, or (iii) at least one agent for modulating the activity, the expression or the maturation of said sequences of the invention, in particular as defined above.

A method or a use of the invention makes it possible to prevent and/or treat skin aging, in particular chronological and/or photoinduced skin aging.

A method or a use of the invention makes it possible to impart and/or restore a youthful appearance to the skin, said appearance being manifested by smooth, elastic, tonic or firm skin, or a luminous complexion.

A method or a use of the invention makes it possible to prevent and/or reduce the presence of a marked microrelief of the skin, in particular at the level of the corner of the lips and of the crow's feet.

A method or a use of the invention makes it possible to reinforce the barrier properties of the skin.

Also described is a cosmetic, or nontherapeutic, method for promoting and/or reinforcing skin moisturization, and preferably for preventing and/or treating dry skin and/or signs of dryness of the skin, in an individual in need thereof, the method comprising at least one step consisting in administering, to said individual, at least one composition comprising, as active agent, (i) at least one amino acid sequence of the invention or (ii) at least one nucleic acid sequence of the invention, or (iii) at least one agent for modulating the activity, the expression or the maturation of said sequences of the invention, in particular as defined above.

A method or a use of the invention also makes it possible to impart and/or restore a moisturized and healthy appearance to the skin, said appearance being manifested by smooth, elastic, tonic or firm skin, or a luminous complexion.

A method or a use of the invention also makes it possible to prevent and/or reduce the presence of squamae, coarseness, cracks, scales and/or wrinkles or fine lines associated with dry skin or skin with a lack of moisturization.

A method or a use of the invention also makes it possible to reinforce the barrier properties of the skin.

Preferably, a method of the invention can comprise the topical application, to at least one part of the skin of an individual in need thereof, in particular to the skin of the face and/or of the neckline, of at least one layer of a topical composition of the invention.

Advantageously, a method of the invention via topical application can comprise the application to the skin, and in particular to the skin of the face, of a composition of the invention in the form of a mask.

A cosmetic method by topical application according to the invention can advantageously comprise the application of a composition of the invention, in combination, simultaneously, successively or separately over time, with an additional cosmetic or dermatological composition distinct from the composition of the invention and intended for caring for and/or making up the skin.

According to another preferred embodiment, a cosmetic method of the invention can be implemented orally, in particular by administration of at least one food or dietetic composition for cosmetic purposes.

According to another preferred embodiment, a cosmetic method of the invention can be implemented parenterally. The parenteral implementation of a cosmetic method of the invention is carried out with the exclusion of any surgical intervention and is merely aimed at performing a surface treatment of the skin for esthetic purposes.

Thus, a cosmetic method of the invention implemented parenterally is carried out by any injection technique or device suitable for an intraepidermal and/or intradermal and/or subcutaneous injection.

Such an administration can be carried out, for example, by mesotherapy.

A cosmetic method implemented parenterally therefore results only in a superficial penetration of the skin and is therefore outside any medical or therapeutic context.

It is alternatively possible, parenterally, to favor administration using a systemic patch.

A cosmetic method according to the invention can be carried out daily, for example at the rate of a single administration per day or of an administration split up into two or three times per day, for example once in the morning and once in the evening.

A cosmetic method according to the invention may be implemented over a time period ranging from one week to several weeks, or even several months, this period moreover possibly being repeated after periods without treatment, for several months or even several years.

By way of example of a cosmetic method according to the invention, it is possible to envision administration of a composition of the invention, for example, at the rate of 1, 2 or 3 times per day, or more, and generally over an extended period of at least 4 weeks, or even 4 to 15 weeks, with, where appropriate, one or more periods of interruption.

Reconstructed Skin

According to another aspect, the present invention relates to the use of an effective amount of at least one amino acid sequence of the invention, or of at least one nucleic acid sequence of the invention, or of at least one modulating agent of the invention, for preparing a multistratified epithelial cell model, preferably a reconstructed skin model.

There is a great advantage to developing organotypic models that are as close as possible to in-vivo conditions for evaluating the safety and efficacy of active agents and formulae for cosmetic and dermatological applications.

The use of at least one amino acid sequence of the invention, or of at least one nucleic acid sequence of the invention, or of at least one modulating agent of the invention, in a culture medium, is capable of improving the quality of the models developed.

A reconstructed skin model according to the invention can be of use for mimicking aged skin, optionally associated with dryness of the skin, or the conditions for restoring the homeostasis of aged skin, optionally associated with dryness of the skin.

According to another aspect, the present invention relates to a method for preparing an isolated multistratified epithelial cell model, and preferably an isolated reconstructed skin, comprising at least the step of bringing at least an effective amount of at least one amino acid sequence, or of at least one nucleic acid sequence, or of at least one modulating agent in accordance with the invention, into contact with cells capable of generating an isolated reconstructed skin, and in particular keratinocytes.

A reconstructed skin model can comprise various cell types, such as keratinocytes, fibroblasts, Langerhans cells and melanocytes. The cells of fibroblast type can optionally be irradiated.

Such models and the preparation thereof are known to those skilled in the art.

According to yet another aspect, the present invention also relates to a method for preparing a multistratified epithelial cell model, preferably a reconstructed skin model, comprising at least one step of culturing cells of at least one cell type of said model, said cells having been genetically modified so as to suppress the expression of an amino acid sequence or of a nucleic acid sequence of the invention.

The production of cells genetically modified so as to suppress the expression of an amino acid sequence or of a nucleic acid sequence of the invention, which cells are referred to as “knock-out”, can be carried out by any method known to those skilled in the art.

By way of example, such cells can be obtained by transfection and homologous recombination of a nucleic acid fragment which inserts into or takes the place of the gene expressing the amino acid sequence of which the expression is to be suppressed.

Likewise, it may be possible to suppress the expression of a given gene by transfection into the cell of a nucleic acid sequence encoding an interfering RNA specific for the mRNA derived from the gene of which the expression is to be suppressed.

### FIGURE LEGEND

FIG. 1: represents the variation in the expression of dermicidin (mean±sem) measured by Western blotting as a function of the degree of moisturization of the skin.

For the purposes of the present invention, “one” should be understood, unless otherwise indicated, in the sense of “at least one”.

The examples and figures hereinafter are presented by way of illustration and without implied limitation of the invention.

### EXAMPLES

### Example 1

### Dermicidin Expression in the Skin

1—Materials and Methods

a—iTRAQ Protocol

The detection of dermicidin (DCD) expression was carried out by means of the iTRAQ (Isobaric Tagging Reagents for Quantitative Proteomic Analysis) method developed by the company Applied Biosystems. This method enables the quantification of peptides prelabeled with an isobaric tag. There are 4 different tags and it is possible to compare up to 4 types of distinct samples in one and the same LC/MS-MS analysis.

The principle of this method is as follows:

—Digestion of Protein Extracts and Labeling:

In a first step, each protein extract is digested with a proteolytic enzyme: trypsin.

In a second step, each peptide mixture obtained is labeled with an isobaric tag having a total mass of 145, comprising a “reporter” group having a mass of 114, 115, 116 or 117, and a “balance” group having a mass of 31, 30, 29 or 28, bonded to one another via an MS fragmentation site.

The labeling is obtained by reaction of the peptide reactive group of each tag with the primary amines of the peptides (N-terminal end of the peptides or the side chain of the lysines or tyrosines).

—LC/MS-MS Analysis and Quantification:

After the labeling step, the samples are pooled and analyzed by mass spectrometry. A given peptide, present in several samples, will have an identical parent mass, but its MS/MS fragmentation spectrum will generate the mass of the reporter group characteristic of each of the iTRAQ reagents. The comparison of the intensity of each reporter ion (114, 115, 116 or 117) then enables the quantification of the peptide.

The iTRAQ method is more precisely described by Zieske (J. Exp. Bot., 2006, 57:1501) or Wiese et al. (Proteomics, 2007, 7:340).

b—Taking and Treatment of Skin Samples

Varnish stripping specimens of stratum corneum having a surface area of 90 cm² are obtained according to the protocol described by Mehul et al., J. Biol. Chem., 2000, 275(17):12841-7, from the external face of the leg. Specimens are taken from two groups of subjects:

1) “Young Skin” Group: 18-40 years old, 27 male subjects;

2) “Aged Skin” Group: over 60 years old, 35 male subjects.

These skin specimens are then analyzed by proteomics by means of the “isobaric labeling” technique previously described, using the iTRAQ Reagents Multiplex kit (4352135), from Applied Biosystems, in accordance with the maker's instructions.

2—Results

Among the proteins identified, the peptides represented by the sequences SEQ ID No.: 17 and SEQ ID No.: 18 derived from dermicidin (accession No. P81605—Ref.: Swissprot/Uniprot) are detected and quantified in four experiments.

The dermicidin quantification results are given in the following table:

| | Experiment No. | | | | | |
|---|---|---|---|---|---|---|
| | 116/114 Aged/young ratio | | | | | Standard |
| Protein | 1 | 2 | 3 | 4 | Mean | deviation |
| dermicidin | 0.3937 | 0.2465 | 0.4356 | 0.4209 | 0.37 | 0.09 |

These results show that the mean aged/young iTRAQ ratio is equal to 0.37±0.09. This measurement reflects a strong expression of dermicidin in the skins of the “Young skin” group and a large decrease in its expression in the skins of the “Aged skin” group.

Dermicidin therefore proves to be an advantageous and specific biomarker for the skin, and more particularly for an aged state of the skin, which may or may not be associated with dryness of the skin.

The expression of apolipoprotein D and of the cathepsin D heavy chain was measured as comparative data.

The results obtained are expressed in the following table.

| | Experiment No. | | | | | |
|---|---|---|---|---|---|---|
| | 116/114 Aged/young ratio | | | | | Standard |
| Protein | 1 | 2 | 3 | 4 | Medium | deviation |
| Apolipo- | 0.7975 | 0.7455 | 0.8205 | 0.9062 | 0.82 | 0.07 |
| protein D | | | | | | |
| Cathepsin D | 0.9580 | 1.1068 | 0.9595 | 0.9537 | 0.99 | 0.07 |
| heavy chain | | | | | | |

In comparison with dermicidin, apolipoprotein D and the cathepsin D heavy chain are only very slightly underexpressed in the “Aged skin” group relative to the “Young skin” group.

This study thus validates the use of dermicidin as an exceptional biomarker for the skin, and more particularly for aged skin, which may or may not be associated with dryness of the skin.

### Example 2

### Dermicidin Expression in the Skin 1—Materials and Methods

a—Western Blotting Protocol

The detection of dermicidin (DCD) expression was carried out by means of a Western blotting method.

The principle of this method is the following: the proteins are separated by electrophoretic migration on a 10-20% Criterion SDS-PAGE gel (Bio-Rad). After semi-dry transfer onto a PVDF membrane, the proteins are incubated in a blocking solution (TBS+0.1% Tween+1% milk), then incubated with the primary antibody directed against the protein of interest overnight at 4° C. A second incubation with the secondary antibody (coupled to a peroxidase) directed against the primary antibody is carried out for one hour at ambient temperature.

Depending on the degree of sensitivity, various kits are used for the visualization: ECL, ECL+ or ECL Advance (Amersham). The image is acquired with a Fluor Smax (Biorad) and the bands are quantified using Quantity-One software (Biorad).

| | | | | | | Calculated |
|---|---|---|---|---|---|---|
| | Nature of | Anti- | Dilu- | Anti- | Detec- | mol. |
| Protein | samples | body I | tion | body II | tion | weight |
| dermicidin | Denatured | Sc- | 1/100 | Anti- | ECL+ | 9 kDa |
| | | 27466/ | | Goat | 60 sec | |
| | | poly | | IgG-HRP | | |

b—Taking and Treatment of Skin Samples

120 female individuals, aged 18 to 70, were selected for this study.

Four zones per leg were delimited (external/internal left leg, external/internal right leg). The samples are taken by varnish stripping (site at the bottom of the leg) according to the protocol described by Mehul et al., (J. Biol. Chem., 2000, 275(17):12841-7).

For this study, the analysis is carried out on one zone: external left leg.

The samples resulting from the varnish strippings were used for the Western blotting analysis. Before the samples were taken, the skin types were characterized by clinical scoring.

The clinical scoring is carried out by a dermatologist according to the “Atlas of dry skin on legs” in order to identify the various skin types according to the degrees below:

0: normal skin, regular skin relief, smooth appearance

1: slightly dry skin, striated skin relief, rough appearance

2: dry skin, striated skin relief and some squamae, rough appearance

3: very dry skin, numerous squamae and some scales, coarse appearance

4: extremely dry skin, numerous scales, coarse appearance.

Preparation of Acetone Powders

Each varnish stripping of 75 cm² is immersed in 75 ml of acetone. The varnish dissolves and the corneocytes are in suspension. The mixture is filtered on a vacuum pump through a 40 μm nylon membrane so as to collect the corneocytes. Two successive washes are carried out with the same volume of acetone. Acetone powders of stratum corneum are obtained in the dry form.

Sample Extraction:

Two types of extraction are carried out successively: native extraction then denaturing extraction.

Native Extraction

The acetone powders are weighed out and placed in 1.5 ml Eppendorf tubes (Trefflab cat. No. 96.07246.9.01). 100 μl of PBS buffer-0.1% triton X100 are added for 1 mg of acetone powder and the mixture is left in contact with ice for one hour. It is then milled for 30 seconds in ice. The medium is then recovered in 0.45 μm Millipore Ultrafree columns and then centrifuged for 5 min at 10,000 g at 4° C.: the supernatant is then collected. A protein assay is carried out according to the BCA technique (Pierce BCA kit). The samples are adjusted to a concentration of 0.15 mg/ml in 4× Laemmli buffer and stored at −20° C. while awaiting analysis.

Denaturing Extraction

It is carried out using the pellet of the native extract. In 1.5 ml Eppendorf tubes (Trefflab cat. No. 96.07246.9.01), 100 μl of 1× Laemmli buffer is added to the pellet for 1 mg of acetone powder. The mixture is boiled for 10 min at 90° C. and then milled for 20 seconds and centrifuged for 10 min at 10 000 g/min. The supernatant is collected. A protein assay is carried out according to the Bradford technique. The samples are adjusted to a concentration of 1 mg/ml and stored at −20° C. while awaiting analysis.

These skin specimens are then analyzed by Western blotting as previously described. The results were obtained from the denatured extracts.

Statistical Analyses

The effect of dryness of the skin (clinical score) on the expression level of the biomarkers was analyzed both graphically, by representing the mean values with their confidence interval as a function of the age classes or of the clinical score of the dryness of the skin, and using a multiple regression model with the age and “dryness of the skin score” parameters as covariables.

The significance of the coefficients (Student's test) associated with the covariables makes it possible to test the association between the biomarker studied and the age and the dryness score. Taking the two parameters into account in the model makes it possible to estimate the effect of one of the parameters while adjusting the effect of the second (all things being otherwise equal).

The level of significance of the tests is fixed at 5% in an unadjusted two-sided approach. The analyses were carried out using the SPPSS software, version 17. The expression levels of the biomarkers were converted beforehand (square root or Log 10) when this was useful for both making their distribution symmetrical and stabilizing their variance.

2—Results

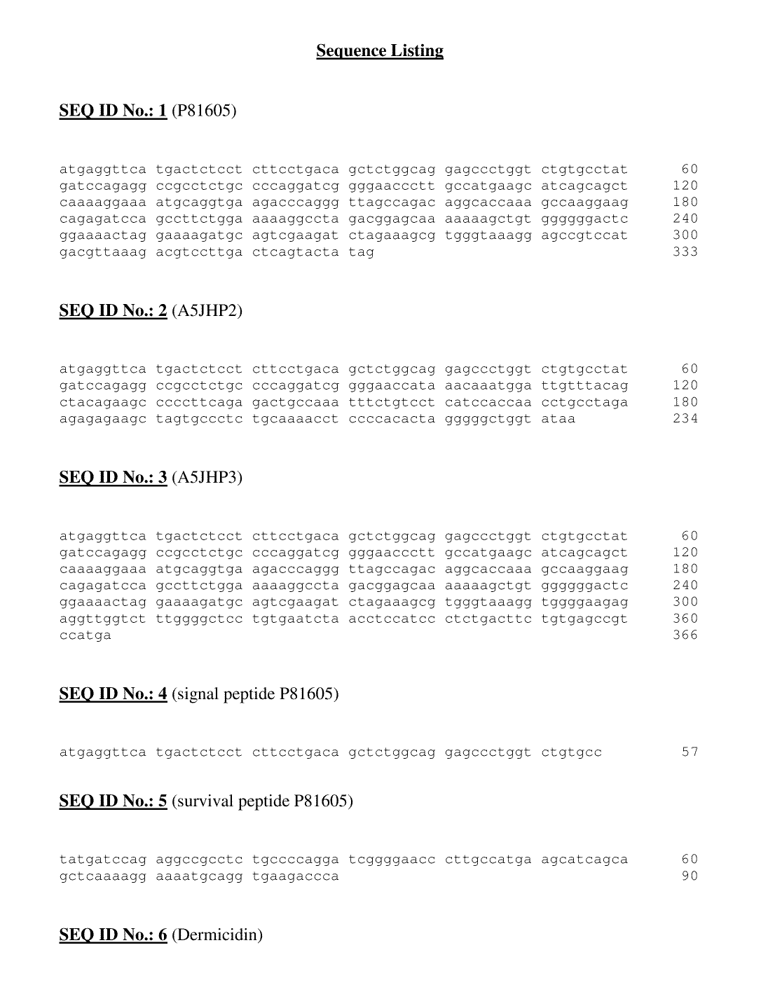

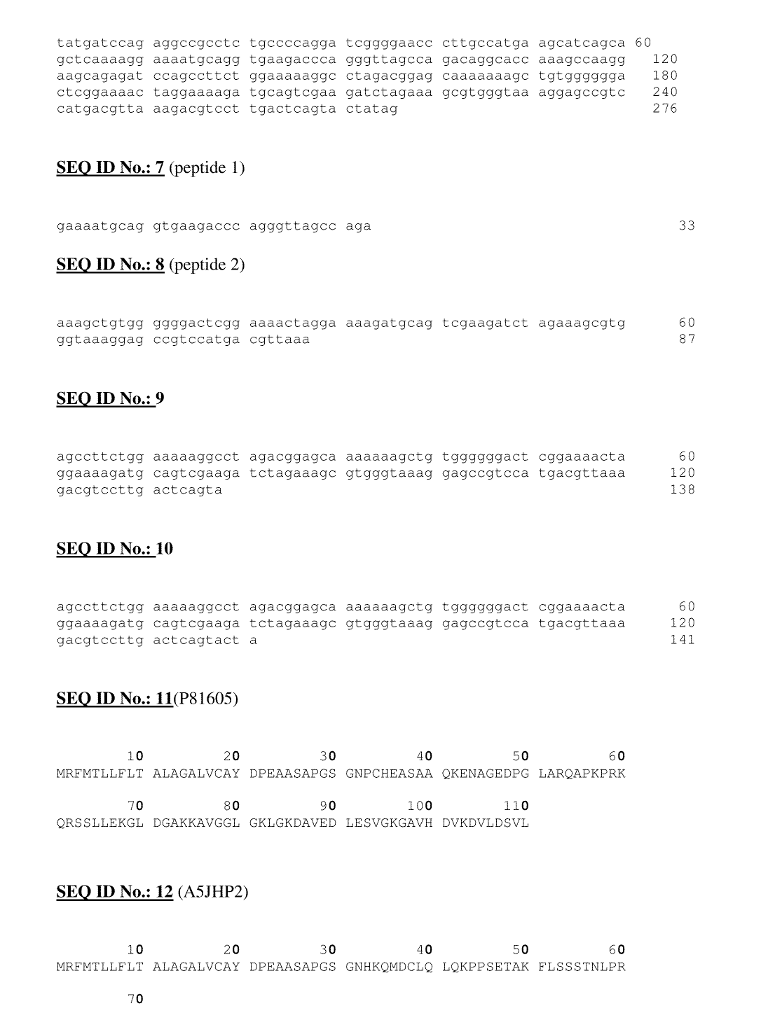

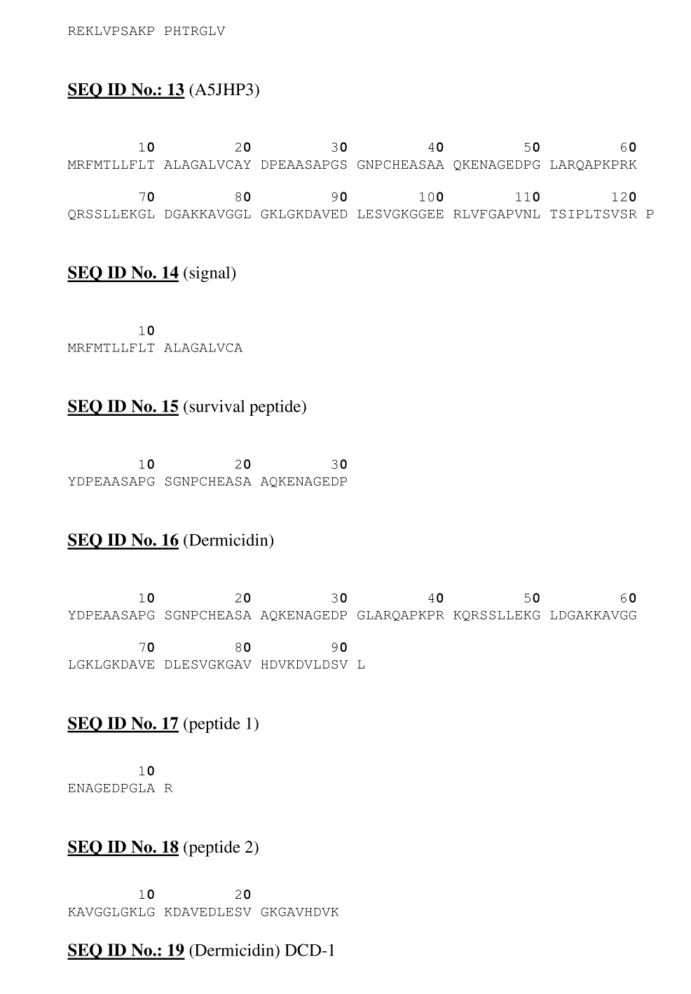

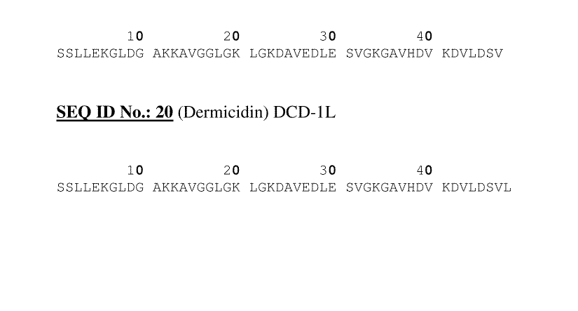

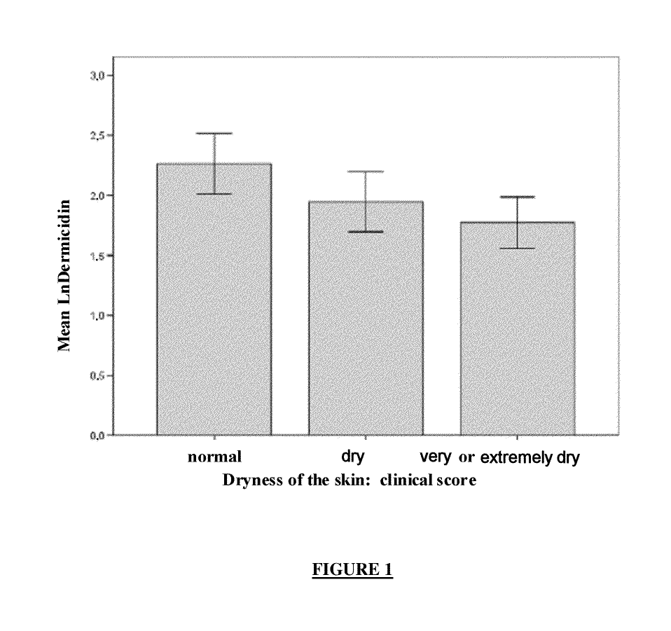

The results obtained, illustrated by FIG. 1, clearly show a decrease in dermicidin expression correlated with an increase in the degree of dehydration, or dryness, of the epidermis.

The statistical analysis confirms a significant decrease in dermicidin expression in dry skin compared with normal skin (p=0.046).

This study thus validates the use of dermicidin as an exceptional biomarker for moisturization of the skin, and more particularly of dry skin.

### Example 3

### Effect of a Bifidobacterium sp. Lysate on Dermicidin Expression

1—Materials and Methods

The skin samples were taken and the dermicidin expression measurements carried out as indicated in example 1.

The volunteers are female subjects 30 to 65 years old. They were combined and divided up into two subgroups according to the treatment administered, a “Placebo” group and a “Treated” group.

The “Treated” group receives topically, on the leg, twice a day, in the morning and in the evening, for 56 days, i.e. 2 months, a composition comprising 10% of Repair Complex® in a carrier formula corresponding to an Arlacel/Myrj oil-in-demineralized water emulsion containing 5% of Parleam, 15% of cyclopentasiloxane, 3% of glycerol and 2% of petroleum jelly.

The Repair Complex® comprises a Bifidobacterium longum lysate registered under the INCI name: Bifida ferment Lysate, under the EINECS name: Bifidobacterium longum, under the EINECS No.: 306-168-4 and under the CAS No.: 96507-89-0. This lysate is sold under the name Repair Complex CLR® by the company K. Richter GmbH.

The “Placebo” group receives, on the other hand, only the carrier formula.

For each “Treated” and “Placebo” subjects condition, a mean of the D56/D0 ratios is obtained, thus making it possible to evaluate the effect of the treatment.

The D56/D0 ratios are calculated according to the following formula:

$$\% effect of the treatment = 100 \times \frac{'' Treated '' D 56 / D 0 Ratio \text{-} '' Placebo '' D 56 / D 0 Ratio}{'' Placebo '' D 56 / D 0 ratio} \\$$

2—Results

The raw data obtained as a result of the analysis are the following:

| | |
|---|---|
| Placebo D56/D0 | Treated D56/D0 |
| Ratio | Ratio |

Experiment 1

| | |
|---|---|
| 2.70 | 3.10 |

Experiment 2

| | |
|---|---|
| 1.00 | 1.62 |

The mean value of the “Placebo” D56/D0 ratio is 1.85, whereas the mean value of the “Treated” D56/D0 ratio is 2.36.

The treatment with the Repair Complex® is reflected by an increase in the signal of 27% compared with the placebo.

Thus, the administration, topically, of a Bifidobacterium longum lysate increases the dermicidin expression by approximately 30% after 2 months of treatment. The administration, topically, of a Bifidobacterium sp. lysate thus makes it possible to restore a dermicidin expression level in the skin, and in particular in aged skin, which may or may not be associated with dryness of the skin, that is favorable to reinforcing its skin protection action, to reinforcing the barrier properties of the skin, and to reducing the signs of skin aging, which may or may not be associated with dryness of the skin.

The administration, topically, of a Bifidobacterium sp. lysate also makes it possible to restore a dermicidin expression level in dry skin that is favorable to reinforcing its skin protection action, to reinforcing the barrier properties of the skin, and to reducing the signs of dryness of the skin.

This study also validates the use of dermicidin as a biomarker for evaluating the efficacy of a cosmetic treatment for aged skin, and also as a tool for screening for new active agents intended for the treatment of aged skin, which may or may not be associated with dryness of the skin.

### Example 4

Effect of a mixture of Bifidobacterium longum lysate and of phytosphingosine salicylate on dermicidin expression

1—Materials and Methods

The skin samples were taken and the dermicidin expression measurements carried out as indicated in example 1.

The volunteers are female subjects 40 to 45 years old. Each subject is their own control; both arms are sampled, but only one is treated with the product tested, and the second arm receives a placebo.

The “Treated” arm receives topically, twice a day, in the morning and in the evening, for 56 days, i.e. 2 months, a composition comprising 10% of Repair Complex® and 0.002% of Phytosphingosine-SLC in a carrier formula corresponding to an Arlacel/Myrj oil-in-demineralized water emulsion containing 5% of Parleam, 15% of cyclopentasiloxane, 3% of glycerol and 2% of petroleum jelly.

The Repair Complex® comprises a Bifidobacterium longum registered under the INCI name: Bifida ferment Lysate, under the EINECS name: Bifidobacterium longum, under the EINECS number: 306-168-4 and under the CAS No.: 96507-89-0. This lysate is sold under the name Repair Complex CLR® by the company K. Richter GmbH.

The phytosphingosine salicylate derivative is the product sold by the company Evonick Goldschmidt under the name Phytosphingosine SLC®.

The “Placebo” arm receives only the carrier formula.

For each “Treated” and “Placebo” condition, a mean of the D56/D0 ratios is obtained as previously indicated, thus making it possible to evaluate the effect of the treatment.

2—Results

The raw data obtained as a result of the experiment are the following:

| | |
|---|---|
| Placebo D56/D0 | Treated D56/D0 |
| Ratio | Ratio |

Experiment No. 1

| | |
|---|---|
| 0.75 | 1.66 |

Experiment No. 2

| | |
|---|---|
| 0.45 | 0.81 |

The mean value of the “Placebo” D56/D0 ratio is 0.6, whereas the mean value of the “Treated” D56/D0 ratio is 1.235.

The treatment effect is equal to 105% and reflects a doubling of dermicidin expression compared with the “Placebo”.

Thus, the administration, topically, of a mixture comprising a Bifidobacterium longum lysate and phytosphingosine salicylate increases dermicidin expression by more than 100%.

The administration, topically, of a mixture comprising a Bifidobacterium longum lysate and phytosphingosine salicylate thus makes it possible to restore a dermicidin expression level in the skin, and in particular in aged skin, which may or may not be associated with dryness of the skin, that is favorable to reinforcing its skin protection action, to reinforcing the barrier properties of the skin, and to reducing the signs of skin aging, which may or may not be associated with dryness of the skin. The administration, topically, of a mixture comprising a Bifidobacterium longum lysate and phytosphingosine salicylate also makes it possible to restore a dermicidin expression level in the skin, and in particular in dry skin, that is favorable to reinforcing its skin protection action, to reinforcing the barrier properties of the skin, and to reducing the signs of dryness of the skin.

### LITERATURE

Anderson, R. K. and W. L. Kenney (1987). << Effect of age on heat-activated sweat gland density and flow during exercise in dry heat<> J. Appl. Physiol. 63(3): 1089-1094.
Baechle, D., T. Flad, et al. (2006). << Cathepsin D is present in human eccrine sweat and involved in the postsecretory processing of the antimicrobial peptide DCD-1L<> J. Biol. Chem. 281(9):5406-5415.
Cunningham, T. J., L. Hodge et al. (1998) << Identification of a survival-promoting peptide in medium conditioned by oxidatively stressed cell lines of nervous system origin<> J. Neurosci 18(18):7047-7060.
Flad, T., R. Bogumil et al. (2002) << Detection of dermcidin-derived peptides in sweat by ProteinChip Technology. <> J. Immunol. Methods 270(1): 53-62.
Kenney, W. L. and S. R. Fowler (1988) << Methylcholine-activated eccrine sweat gland density and output as a function of age<> J. Appl. Physiol. 65(3):1082-1086.
Kyte et al., (1982), J. Mol. Biol., 157: 105.
Mehul B. et al., (2000) << Identification and Cloning of a New Calmodulin-like Protein from Human Epidermis<> J. Biol. Chem. 275(17):12841-12847
Moreira, D. F, B. E. Strauss et al. (2008) << Genes up- and down-regulated by dermcidin in breast cancer: a microarray analysis<> Genet. Mol. Res. 7(3): 925-932.
Niyonsaba, F. A. Suzuki et al. (2009) << The human antimicrobial peptide dermcidin activates normal human keratinocytes<> Br. J. Dermatol. 160(2): 243-249.
Rieg, S. C., Garbe et al. (2004) << Dermcidin is constitutively produced by eccrine sweat gland and is not induced in epidermal cells under inflammatory skin conditions<> Br. J. Dermatol. 151(3): 534-539.
Rieg S., II Steffen et al. (2005) << Deficiency of dermcidin-derived antimicrobial peptides in sweat of patients with atopic dermatitis correlates with an impaired innate defense of human skin in vivo<> J. Immunol. 174(12):8003-8010.
Sakurada K., T. Akutsu, et al., (2009) << Detection of dermcidin for sweat identification by real-time RT-PCR and ELISA<> Forensic. Sci. Int. 2010 Jan. 30; 194(1-3):80-4. Epub 2009 Nov. 13.
Sambrook et al., 1989, Vol. I-III, Coldspring Harbor Laboratory, Coldspring Harbor Press, NY.
Schittek, B, R. Hipfel et al. (2001): << Dermcidin: a novel human antibiotic peptide secreted by sweat glands<> Nat. Immunol. 2(12): 1133-11137.
Watchorn, T. M. N. Dowidar et al. (2005) << The chachectic mediator proteolysis inducing factor activates NF-kappaB and STAT3 in human Kupffer cells and monocytes<> Int. J. Oncol. 27(4): 1105-1111.
Wiese et al., (2007) << Protein labeling by iTRAQ: A new tool for quantitative mass spectrometry in proteome research<> Proteomics 7(3):340-350
Zieske et al. (2006) << A perspective on the use of iTRAQ reagent technology for protein complex and profiling studies<> J. Exp. Bot. 57(7):1051-1058.

1. The use (i) of at least one amino acid sequence encoded by a nucleic acid sequence represented by SEQ ID No.: 1, or of an analog or fragment of said amino acid sequence, or (ii) of said nucleic acid sequence, as a biomarker for a state of aged skin and/or for the signs of skin aging, which may or may not be associated with dryness of the skin.

2. The use as claimed in claim 1, wherein said nucleic acid sequence is represented by a sequence chosen from SEQ ID No.: 2 to SEQ ID No.: 10, or an analog or fragment thereof.

3. The use as claimed in claim 1, wherein said amino acid sequence is represented by a sequence chosen from SEQ ID No.: 11 to SEQ ID No.: 20, or an analog or fragment thereof, and is preferably chosen from SEQ ID No.: 16, SEQ ID No.: 17, SEQ ID No.: 18, SEQ ID No.: 19, SEQ ID No.: 20, or an analog or fragment thereof.

4. The use as in claim 1, wherein a decrease in the activity, in the expression or in the maturation of said biomarker is indicative of aged skin and/or of signs of skin aging, which may or may not be associated with dryness of the skin.

5. The use (i) of at least one amino acid sequence chosen from SEQ ID No.: 12, SEQ ID No.: 13, SEQ ID No.: 14, SEQ ID No.: 15, SEQ ID No.: 16, SEQ ID No.: 17, SEQ ID No.: 18, SEQ ID No.: 19, SEQ ID No.: 20, an analog having a sequence identity of at least 90% with said sequence and a biological activity of the same nature, or a fragment thereof which comprises from 3 to 48 amino acids of said sequence and which has a biological activity of the same nature, or (ii) of at least one nucleic acid sequence as defined in claim 1, for screening for active agents or physical treatments capable of modulating the activity, the expression or the maturation of said amino acid sequence or of said nucleic acid sequence.

6. The use (i) of at least one amino acid sequence as defined in claim 1, or (ii) of at least one nucleic acid sequence as defined in claim 1, for screening for active agents or physical treatments for the care of aged skin and/or the prevention and/or treatment of the signs of skin aging, which may or may not be associated with dryness of the skin, capable of modulating the activity, the expression or the maturation of said amino acid sequence or of said nucleic acid sequence.

7. The use (i) of at least one amino acid sequence as defined in claim 1, or (ii) of at least one nucleic acid sequence as defined in claim 1, for characterizing the efficacy of a cosmetic treatment for the skin.

8. The use as claimed in claim 7, wherein the cosmetic treatment is a cosmetic treatment for aged skin and/or for the signs of skin aging, which may or may not be associated with dryness of the skin.

9. The cosmetic use of an effective amount (i) of at least one amino acid sequence as defined in claim 1, or (ii) of at least one nucleic acid sequence as defined in claim 1, as an active agent for preventing and/or treating aged skin and/or the signs of skin aging, which may or may not be associated with dryness of the skin.

10. The use as claimed in claim 1, wherein the signs of skin aging are chosen from thinning of the skin, a loss of firmness, a loss of elasticity, a loss of density or a loss of tonicity of the skin, dryness of the skin, the appearance of a marked microrelief of the skin, the formation and/or the presence of fine lines and/or of wrinkles, a modification of the radiance of the skin complexion, a wizened appearance of the skin, a modification of the odor of the skin, sagging of the skin and withering of the skin.

11. The use as claimed in claim 1, wherein the signs of dryness of the skin are chosen from withered skin, a lack of elasticity, of suppleness and/or of tonicity of the skin, a coarse feel, the presence of cracks, a desquamation, the presence of scales, wrinkles and fine lines associated with dry skin, and a feeling of tautness and/or itching.

12. The use of an effective amount (i) of at least one amino acid sequence as defined in claim 1, or (ii) of at least one nucleic acid sequence as defined in claim 1, or of at least one agent for modulating the activity, the expression or the maturation of said amino acid sequence or of said nucleic acid sequence, for preparing a multistratified epithelial cell model, preferably a reconstructed skin model.

13. An in vitro or ex vivo method for characterizing a state of aged skin and/or the signs of skin aging, which may or may not be associated with dryness of the skin, comprising at least the steps consisting in:
a) carrying out, in an isolated sample of skin, a qualitative or quantitative measurement of the expression, the maturation or the activity of said amino acid sequence as defined in claim 1 or of said nucleic acid sequence as defined in claim 1, and
b) comparing said measurement carried out in step a) to a reference measurement.

14. The use as claimed in claim 1, or the method as claimed in claim 13, wherein the aged skin is chosen from skin having undergone chronological aging and/or photoinduced aging.

15. An in vitro or ex vivo method for screening for active agents or physical treatments capable of modulating the activity, the expression or the maturation of an amino acid sequence chosen from SEQ ID No.: 12, SEQ ID No.: 13, SEQ ID No.: 14, SEQ ID No.: 15, SEQ ID No.: 16, SEQ ID No.: 17, SEQ ID No.: 18, SEQ ID No.: 19, SEQ ID No.: 20, an analog having a sequence identity of at least 90% with said sequence and a biological activity of the same nature, or a fragment thereof comprising from 3 to 48 amino acids of said sequence and having a biological activity of the same nature, or of a nucleic acid sequence as defined in claim 1, comprising at least the steps consisting in:
a) placing said amino acid sequence or said nucleic acid sequence under conditions favorable to the activity, the expression or the maturation of said sequences,
b) bringing said amino acid sequence or said nucleic acid sequence into contact with at least one active agent to be tested, or exposing said amino acid sequence or said nucleic acid sequence to a physical treatment to be tested,
c) carrying out a qualitative or quantitative measurement of the expression, the maturation or the activity of said amino acid sequence or of said nucleic acid sequence, and
d) comparing said measurement to a reference measurement.

16. An in vitro or ex vivo method for screening for active agents or physical treatments for the care of aged skin and/or the prevention and/or treatment of the signs of skin aging, which may or may not be associated with dryness of the skin, capable of modulating the activity, the expression or the maturation of an amino acid sequence as defined in claim 1, or of a nucleic acid sequence as defined in claim 1, comprising at least the steps consisting in:
a) placing said amino acid sequence or said nucleic acid sequence under conditions favorable to the activity, the expression or the maturation of said sequences,
b) bringing said amino acid sequence or said nucleic acid sequence into contact with at least one active agent to be tested, or exposing said amino acid sequence or said nucleic acid sequence to a physical treatment to be tested,
c) carrying out a qualitative or quantitative measurement of the expression, the maturation or the activity of said amino acid sequence or of said nucleic acid sequence, and
d) comparing said measurement to a reference measurement.

17. A cosmetic method for preventing and/or treating aged skin and/or signs of skin aging, which may or may not be associated with dryness of the skin, in an individual in need thereof, comprising at least one step consisting in administering, to said individual, at least one composition comprising, as active agent, (i) at least one amino acid sequence as defined in claim 1, or (ii) at least one nucleic acid sequence as defined in claim 1.

18. An in vitro or ex vivo cosmetic method for characterizing the efficacy of a cosmetic treatment for the skin in an individual in need thereof, comprising at least the steps consisting in:
a) carrying out, before the implementation of the cosmetic treatment, in a first isolated skin sample taken from said individual, at least one first qualitative or quantitative measurement of the expression, of the maturation or of the activity of at least one amino acid sequence as defined in claim 1 or of at least one nucleic acid sequence as defined in claim 1,
b) carrying out, after the implementation of the cosmetic treatment, in a second isolated skin sample taken from said individual, at least one second qualitative or quantitative measurement of the expression, of the maturation or of the activity of said amino acid sequence or of said nucleic acid sequence, and
c) comparing the first and second measurements, in particular in order to deduce therefrom information relating to at least one effect of the implementation of the cosmetic treatment.

19. An isolated peptide represented by an amino acid sequence chosen from SEQ ID No.: 18, or an analog or fragment thereof.

20. A cosmetic composition comprising a peptide as defined in claim 19 or a nucleic acid sequence encoding such a peptide.
